(19) United States
(12) Patent Application Publication
(10) Pub. No.: US 2022/0135923 A1
(43) Pub. Date: May 5, 2022

(54) DYNAMIC POLYMER SURFACES FOR SCREENING, ENRICHMENT, AND HARVESTING OF CELLS AND OTHER SOFT COLLOIDAL PARTICLES

(71) Applicant: University of Georgia Research Foundation, Inc., Athens, GA (US)

(72) Inventors: Yongwook Kim, Athens, GA (US); Amine Laradji, St. Louis, MO (US); Sergiy Minko, Athens, GA (US); Nataraja Sekhar Yadavalli, Athens, GA (US)

(21) Appl. No.: 17/514,795

(22) Filed: Oct. 29, 2021

Related U.S. Application Data
(60) Provisional application No. 63/107,785, filed on Oct. 30, 2020.

(51) Int. Cl. C12M 1/12; G03F 7/38; G03F 7/32; G03F 7/038; G03F 7/16; G03F 7/20; C12M 1/32; C12M 1/42; C12M 1/26; C12N 1/02; C12N 1/04; C08L 33/26

(52) U.S. Cl. CPC C12M 25/08; C12N 2533/30; G03F 7/325; G03F 7/0382; G03F 7/162; G03F 7/2004; C12M 23/12; C12M 35/02; C12M 33/00; C12N 1/02; C12N 1/04; C08L 33/26; C12N 2513/00; C12N 2529/00; C12N 2539/10; C12N 2535/10; G03F 7/38

(57) ABSTRACT

Dynamic polymer surfaces are provided that include alternating micropatterns of adhesive domains and environmental stimuli-responsive repulsive domains, where application of a select environmental stimulus activates polymer structures of the repulsive domains to change conformation with respect to the adhesive domains. The dynamic polymer surfaces are useful for sorting, screening, and enriching target particles (such as cells) in a sample and for culturing and harvesting cells. Products, such as cell culture systems, including the dynamic polymer surfaces are also provided.

### CROSS-REFERENCE RELATED APPLICATION

This application claims the benefit of U.S. Provisional Application entitled “DYNAMIC POLYMER SURFACES FOR SCREENING, ENRICHMENT, AND HARVESTING OF CELLS AND OTHER SOFT COLLOIDAL PARTICLES,” having Ser. No. 63/107,785, filed on Oct. 30, 2020, which is incorporated herein by reference in its entirety.

### STATEMENT REGARDING FEDERALLY SPONSORED RESEARCH OR DEVELOPMENT

This invention was made with Government support under Grant No. 1904365, awarded by the National Science Foundation. The Government has certain rights in the invention.

### BACKGROUND

A large global market exists for cell therapy processing. Currently, there are over 150 cell therapy start-ups to mid-sized companies in United States with many cell therapy products pipelined and going through clinical trials. Due to health conditions and diseases, such as the COVID-19 pandemic, the need for cellular therapies for organ damage/failure have increased tremendously. A bottleneck of this industry is cell therapy processing, involving scale-up or scaled-out biomanufacturing for allogenic and autologous cell therapies, respectively. Many of these companies must scale adherent cell cultures on supporting matrices which mimic extracellular microenvironments. Some are using alternatives such as microcarrier beads to grow adherent cell cultures and suffering with cell harvesting and sorting problems. There is also a great need for fast, efficient, and effective methods for quickly screening for and enriching rare cells and/or other target particles from a sample without time-consuming labeling steps or low-specificity size-based sorting methods.

Existing cell manufacturing technologies for culturing adherent cells includes the steps of seeding, culture, detachment, and collection of cells. The detachment of the confluent monolayer of cells is an essential step, which is currently achieved by the standard method of trypsinization. Enzymatic (trypsin) harvesting of cells is discouraged by FDA guidelines due to cellular surface receptor damage and risk of toxicity to the patients due to viral vectors from animal sourced trypsin, and this method also suffers from some economic hurdles. EDTA based non-enzymatic cell harvesting also has efficacy issues, as cell damage is a persistent problem. Additionally, effective methods for screening samples, cell populations, and other samples for specific and/or rare target particles and/or cells without the need for time-consuming labeling steps or the use of reagents that are potentially damaging to the target particles/cells are also in high demand. Thus, in order to grow and harvest large quantities of cells needed for successful cell therapies and to provide efficient and effective screening/enriching technologies, the industry is searching for an effective system incorporating both effective cell growth and safe, non-damaging, non-enzymatic cell harvesting as well as solutions for sorting/enriching target particles in samples.

### SUMMARY

In accordance with the purpose(s) of the disclosure, as embodied and broadly described herein, the disclosure, in various aspects, relates to dynamic polymer surfaces with alternating micropatterns of adhesive and repulsive domains, products including the dynamic polymer surfaces, and methods of making the dynamic polymer surfaces. The methods of the present disclosure also provide methods of using the dynamic polymer surfaces of the present disclosure for isolation, separation, and/or enrichment of particles from a sample, as well as for screening for cell populations from samples and methods of culturing and harvesting cells.

Aspects of the present disclosure provide dynamic polymer surfaces with a polymer layer having alternating micropatterns of adhesive domains and environmental stimuli-responsive repulsive domains. In embodiments the adhesive domains including one or more first polymer structures and configured to have affinity for a target soft colloid particle. In embodiments, the repulsive domains including one or more second polymer structures that change form a retracted conformation to a swollen conformation in response to an environmental stimulus, such that application of the environmental stimulus activates the second polymer structures to the swollen conformation and does not activate the first polymer structures to a swollen conformation, such that the repulsive domains enlarge with respect to the adhesive domains. Embodiments of the present disclosure also include products including a substrate coated with the dynamic polymer surface of the present disclosure, such as products like a cell culture system that may also include a controlled environment in which the coated substrate is housed.

The present disclosure also includes methods for non-enzymatically detaching particles from a polymer surface of the present disclosure. In embodiments, the method includes the steps of:

a) contacting the dynamic polymer surface of the present disclosure with a liquid composition including target soft colloid particles with affinity for the adhesive domains in a controlled environment in for a first period of time during which the repulsive domains are in the retracted conformation; and

b) applying an activating environmental stimulus to the controlled environment for a second period of time to activate the second polymer structures to the swollen conformation such that the repulsive domains enlarge with respect to the adhesive domains, thereby physically contacting and exerting a mechanical force on particles adhered to the adhesive domains sufficient to detach a portion of particles from the adhesive domains.

Methods of the present disclosure can further include the steps of:

c) removing the activating environmental stimulus for a third period of time, such that the repulsive domains return to the retracted confirmation; and

d) repeating steps b and c for a number of cycles effective to increase the percent of target soft colloid particles bound to the adhesive domains.

In yet additional embodiments, the method can further include the steps of:

e) removing the liquid composition from the controlled environment to retain bound target soft colloid particles on the dynamic polymer surface; and

f) releasing bound target soft colloid particles from the adhesive domains to obtain an enriched sample of target soft colloid particles.

Other systems, methods, devices, features, and advantages of the devices and methods will be or become apparent to one with skill in the art upon examination of the following drawings and detailed description. It is intended that all such additional systems, methods, devices, features, and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic illustration of an embodiment of synthesis of patterned polymer brush surface of alternating PNIPAM and PtBA domains. FIG. 1 shows the steps of, PGMA coating (1)/(2), Attachment of photo-DIBO at 53° C. overnight (3), applying UV light (365 nm) (4), immobilization of Azido-Bib initiator (4), grafting PNIPAM by ARGET-ATRP (6), NaN³(7) and immobilization of Azido-Bib initiator on previously protected areas (8), grafting PtBA by ARGET-ATRP (9), hydrolysis of PtBA to PAA (10/11); and RGD conjugation (12).

FIGS. 2A-D illustrate a schematic of an embodiment of the dynamic polymer surface of the present disclosure for isolation/enrichment of target particles. FIGS. 2A-2C illustrates the microstructured polymer brush layer made of adhesive domains that carry affinity motifs and repulsive domains made of thermoresponsive polymer in aqueous medium. At T>T_{LCST} the repulsive domain are collapsed and colloids interact only with adhesive domains (FIG. 2A); at T<T_{LCST} the repulsive domains are swollen and cause the mechanical force to detach colloids (FIG. 2A); and after multiple detachment-binding cycles the solution-adsorbent system reaches the affinity-based equilibrium (FIG. 2C). FIG. 2D schematically illustrates an embodiment of a closed system in which a microstructured polymer brush decorated plate (2) is mounted in a liquid flow cell with a thermoconductive bottom wall (1) where temperature is controlled by a thermocouple (3). FIG. 2E is a graph showing experimentally measured periodic change of temperature in the cell between temperatures above and below LCST.

FIGS. 3A-J illustrate AFM surface topography images (the error of the thickness estimation is ±5 nm), cross-sectional profiles, and properties for the embodiments of microstructured polymer structures of the present disclosure. FIGS. 3A-3D illustrate a one-component PNIPAM brush: FIGS. 3A, 3B in aqueous medium at T<T_{LCST}; FIGS. 3C, 3D in aqueous medium at T>T_{LCST}; FIGS. 3E, 3F two-component PNIPAM-RGD-PAA domains in the dry state. FIGS. 3G-3J illustrate a two-component polymer brush in aqueous medium with RGD-PAA domains grafted in-between PNIPAM domains: FIGS. 3G, 3H at T<T_{LCST} when PNIPAM domains are swollen and thicker than RGD-PAA domains; FIGS. 31, 3J at T>T_{LCST} when PNIPAM domains are collapsed and thinner than RGD-PAA domains. FIG. 3K is a graph illustrating temperature dependence swelling of PNIPAM brushes of different thicknesses.

FIGS. 4A-4J illustrate fluorescent microscopy images and graphs of 3T3 cells binding at T>T_{LCST} (FIGS. 4A, 4C, 4E, and 4G) and detachment at T<T_{LCST} (FIGS. 4B, 4D, 4F, and 4H) at the microstructured stimuli-responsive polymer brush. FIGS. 4A and 4B are a control experiment when the cells are bound to one-component RGD-PAA brush only; FIGS. 4C-4H represent a two-component brush with different thickness ratios of PNIPAM collapsed (PNIPAM_{C}) and swollen (PNIPAM_{S}) to RGD-PAA domains as shown in corresponding columns of the panel in FIG. 4I; the error of the thickness estimation is ±5 nm. FIGS. 4E, 4F shows the most efficient binding-detachment when the collapsed PNIPAM_{C} domains are thinner than RGD-PAA domains at T>T_{LCST} and thicker than RGD-PAA domains at T<T_{LCST}; FIGS. 4G, 4H illustrate the case of a PNIPAM brush which is thicker than RGD-PAA even above LCST—the cells attach only to the PNIPAM brush and remain adhered in all the temperature range; FIG. 4J illustrates fractions of retained cells after cooling below LCST. Scale bar (50 μm).

FIGS. 5A-5B illustrate assessment of adhesive F_{A} and repulsive F_{B} forces of interaction between colloidal particles and adhesive and repulsive domains of the microstructure stimuli-responsive polymer brush at T<T_{LCST} for spherical (labeled with s subscripts) and disk-like (labeled with d subscripts) particles: as a function of particle diameter (FIG. 5A) and for different ratios of surface areas of repulsive (Rₛ, R_{d}) and adhesive (Aₛ, A_{d}) domains of the brush (FIG. 5B). The horizontal dash line in FIG. 5B marks F_{B}/F_{A}=1.

FIGS. 6A-6F illustrate isolation of U-87MG cancer cells for different ratios U-87MG: blood cells: 1:10⁵ (FIG. 6A), 1:5×10⁷ (FIG. 6B), and 1:109 (FIG. 6C). FIG. 6D is a graph illustrating the fraction of recovered cells (with an accuracy of 95%) as a function of time and number of temperature cycles. FIGS. 6E and 6F are images illustrating expansion of the recovered RFP-B16F10 cells, with FIG. 6E showing the image of adhered cells after the isolation, and FIG. 6F showing after 6-days of proliferation. Scale bar (50 μm).

FIG. 7A illustrates a schematic of the synthesis of an embodiment of a microstructured polymer interface of the present disclosure, illustrating the following steps: 1,2-Cleaned Si-wafer in Piranha solution was functionalized with APTES, followed by the immobilization of BIBB, ATRP initiators; 3,4-adhesive domains made of SU-8 photoresist were fabricated on the surface under the photomask by UV light; 5-SU-8 film was baked on the hotplate and developed in SU-8 development solution to produce the patterns; 6-NIPAM monomers were polymerized by ARGET-ATRP from the surface initiated substrate; 7-cell adhesive motif, RGD, was conjugated on the surface of SU-8 domains. FIG. 7B illustrates fabrication of master.

FIGS. 8A-8I illustrate AFM surface topography images (the error of the thickness estimation is ±5 nm) and cross-sectional profiles for the microstructured polymer brushes: FIGS. 8A-8C PNIPAM-SU-8 in dry state, FIGS. 8D-8F in aqueous medium at T<T_{LCST} when PNIPAM domains are swollen and thicker than SU-8 domains, FIGS. 8G-8I in aqueous medium at T>T_{LCST} when PNIPAM domains are collapsed and thinner than SU-8 domains.

FIGS. 9A-9F illustrate proliferation and detachment of RAW264.7 cells on PS coverslip, PNIPAM monolayer, and PNIPAM-RGD@SU-8 interface. FIGS. 9A-9B illustrate proliferated RAW264.7 cells on PNIPAM monolayer and PNIPAM-RGD@SU-8 before detachment; FIGS. 9C, 9D show remaining cells on the surface after temperature oscillated cell detachment; FIG. 9E is a graph showing detachment efficiency (%); and FIG. 9F is a graph illustrating the number of cells grown on each interface.

FIGS. 10A-10G illustrate the evaluation of cells through viability testing, f-acitin assay, and reattachment test. FIG. 10A is a graph of MTT assay results, FIG. 10B is a graph of Alamar Blue assay results. In the images of FIGS. 10C and 10D, red fluorescent signal indicates f-actin area and blue signal indicate nucleus of the cell, (cells in FIG. 10C detached with conventional trypsinization and cells in FIG. 10D detached with the proposed method). FIG. 10E is a graph of surface area of cells detached from PS coverslip by conventional trypsinization vs. from the PNIPAM-RGD dynamic surface of the present disclosure by the proposed method. FIGS. 10F and 10G illustrate reattachment of cells previously detached by conventional method (FIG. 10F) and the proposed method (FIG. 10G).

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of biochemistry, material science, molecular biology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20-25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

It must be noted that, as used in the specification and the appended claims, the singular forms “a,” “an,” and “the” include plural referents unless the context clearly dictates otherwise. Thus, for example, reference to “a cell” includes a plurality of cells. In this specification and in the claims that follow, reference will be made to a number of terms that shall be defined to have the following meanings unless a contrary intention is apparent.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### Definitions

In describing and claiming the disclosed subject matter, the following terminology will be used in accordance with the definitions set forth below.

“Polymer” is any natural or synthetic molecule that can form long molecular chains, such as polyesters, polyamides, polyolefins, polystyrene, polyacrylates, polycaprolactone, poly(ethylene oxide), polyurethanes, and other synthetic polymers and biopolymers (macromolecules) including polypeptides, polysaccharides, DNA, and blends of these.

As used herein, the term “soft colloid particle,” “soft colloidal particle” or “soft colloid” includes deformable, membraned particles, hydrogel particles such as, but not limited to, lipid vesicles, cells (e.g., living cells (such as mammalian cells) or dead cells), cellular organelles, polymer capsules, protein complexes, microgel particles, and the like. Soft colloids/soft colloid particles are sometimes abbreviated herein as “SC” and/or “SCPs” as appropriate.

As used herein, the term “biocompatible,” with respect to a substance or fluid described herein, indicates that the substance or fluid does not adversely affect the short-term viability or long-term proliferation of a target biological particle within a particular time range.

As used herein, the term “biological sample” refers to a sample obtained from a biological organism (e.g., a living subject such as a plant, animal, including mammals, and particularly humans), made with material obtained from a biological organism or including a biological organism (e.g., single-celled organisms, such as bacteria, etc.). Example biological samples can include, but are not limited to, whole blood, plasma, urine, saliva, biological secretions, biopsy samples, tissue samples in a biocompatible medium, in vitro expanded cells, and the like, and combinations of these.

### DISCUSSION

In accordance with the purpose(s) of the present disclosure, as embodied and broadly described herein, embodiments of the present disclosure, in some aspects, relate to dynamic polymer surfaces for non-enzymatic release of particles (e.g., soft colloid particles, such as but not limited to, cells, vesicles, and the like) coupled to (e.g., bound, adhered, attached, or otherwise physically connected to) the surface. The present disclosure also includes cell sorting/enrichment systems and cell culture and harvesting systems including the dynamic polymer surfaces of the present disclosure. The present disclosure also includes methods of making the dynamic polymer surfaces as well as methods of using the dynamic polymer surfaces for non-enzymatically detaching particles, such as soft colloid particles. Methods of the present disclosure also include methods of sorting/separating target soft colloid particles from a liquid composition (e.g., a biological sample) and/or enriching the target soft colloid particles in the sample. The present disclosure also includes methods of culturing, growing, and harvesting of target cells to provide cell populations useful for applications such as cell therapies, tissue engineering, and the like.

As discussed above, there is a need in the industry for technologies and systems to allow scale-up of cell culturing and harvesting processes in order to provide the quantities of cell populations needed for various cell-based therapies. Most industrial cell cultures originate from animal or human tissues and can proliferate in adherent or non-adherent culture systems. Non-adherent cells can be effectively cultured in large scale industrial reactors with precise control of growth media, temperature, pH, and other parameters, usually leading to a large yield of cells or their metabolites. Adherent cell cultures require specially prepared cell-adhesive substrates to attach in order to proliferate and maintain their normal physiological activity.

Upon reaching confluency, these cells are harvested using various detachment techniques, the most common of which is called trypsinization which involves addition to cell culture flask small amounts of the protease trypsin (first introduced in 1916 by Rous and Jones), usually in combination with chelating agent EDTA. These two components working together, can effectively accomplish detachment of cells; EDTA binds to calcium, an essential ion for anchoring proteins cadherins, while trypsin cuts through the proteins of the ECM, thus chemically removing the binding filaments. The trypsinization isn't limited just to the ECM proteins, the protease effectively lyses other cell surface proteins, some of which will require 24-48 hours to be re-expressed, while others will be lost permanently. Furthermore, there are also regulatory concerns due to the origin of trypsin (such as porcine small intestine), which hinders its applications for tissue engineering and regenerative medicine.

While trypsinization remains the most widely used detachment method, various non-enzymatic treatments have been developed, each of them having their own benefits and limitations. These methods include mechanical stimulations, such as, scraping or shaking, physical actions (e.g., thermal, light, magnetic or electrical), among others. Overall, the regenerative medicine field is seeking an effective closed system incorporating cell growth and non-enzymatic cell harvesting and cell sorting solutions. There do not appear to be any currently successful alternatives to address these harvesting challenges.

In the alternative, stimuli-responsive cell attachment and detachment approaches such as thermoresponsive, pH, photoresponsive, gel-sol transition substrates are being developed for detachment of adherent cells or cell sheets for tissue engineering applications. One such technology is based on polymer films made of thermo responsive polymers, such as poly(N-isopropylacrylamide) (PNIPAM). PNIPAM-based thermoresponsive culture plates, which employ a continuous thermoresponsive polymer layer, were commercialized and are available for purchase, for example, via Millipore Sigma (Nunc® UpCelI™ Surface cell culture dish). However, this technology did not find wide commercial applications because of the high costs (about $37 for a dish with a cell culture area of 21.5 cm²). These high costs are due in part to a complex fabrication method that involves electron beam initiated polymerization of the monomer on the surface of the support (typically polystyrene, PS). It was found that only surface grafted polymer films are efficient in cell adhesion and detachment. An additional complication is that the cells adhere and detach only on PNIPAM films with an optimal thickness, which depends on the substrate. For example, the determined optimal thickness is 5-30 nm for tissue culture polystyrene (TCPS) wells, but it was found to be only 3.5 nm on glass surfaces.¹². This sensitivity of the film performance to the thickness has a substantial impact on the costs of manufacturing. The effect of film thickness is not fully understood, but it may be related to the intricate balance of the PNIPAM film interactions with cell membrane ingredients and extracellular matrix protein adsorbed on the PNIPAM surface and surface morphology, which is impacted by mechanical properties of the substrate. This delicate balance is difficult to control for scaled-up applications.

Unlike previous approaches using uniform stimuli-responsive coatings, the present systems and methods are based on a microstructured stimuli-responsive polymer film with alternating adhesive and repulsive domains for a dynamic, rather than static/uniform surface. However, in contrast to various approaches using homogeneous stimuli-responsive polymer films, the approach of the surfaces, films, systems, and methods of the present disclosure employs micropatterned surfaces with adhesive and stimuli-activated (e.g., temperature-activated, pH activated, chemo-activated) repulsive domains. This approach overcomes many of the drawbacks of the uniform thermo-responsive surfaces that demonstrate either a weak cell adhesion, poor cell detachment efficiency, or both. The adhesion and detachment forces of those uniform films rely on the same material undergoing phase transition with temperature change, while the interaction of the surface with cells depends on additional factors, such as polymer interactions with water, ECM components, film thickness, substrate, and cell culture. The micropatterned, dynamic surfaces of the present disclosure with distinct domains achieves both high adhesion and high efficiency (and safety) of cell detachment regardless of the film thickness and type of substrate, because adhesive and repulsive properties can be adjusted for each domain independently.

As herein described, the present disclosure thus provides dynamic micropatterned polymer surfaces with alternating adhesive domains and environmental stimuli-responsive repulsive domains, systems including the dynamic polymer surfaces, methods of making the surfaces, and methods of using the dynamic polymer surfaces for detachment of particles from surfaces, for use in screening/selecting and enriching target soft colloid particles, and for culture growth and harvesting of target cells.

Dynamic Polymer Surfaces

Embodiments of dynamic polymer surfaces of the present disclosure include a surface (e.g., surface layer, surface coating, etc. on a surface of a substrate) including a polymer layer having alternating micropatterns of adhesive domains and environmental stimuli-responsive repulsive domains. The polymer layer is made of polymer structures such as, but not limited to polymer brushes (e.g., a polymer layer having polymerized, finger-like projections of polymers providing a “brush” morphology like the bristles of a bush), grafted polymers, anchored polymers, a polymer network, a polymer hydrogel thin film, and combinations thereof. In embodiments the polymer structures of the polymer layer may be of a different type and/or conformation in different domains of the surface.

The polymer layer of the dynamic polymer surfaces of the present disclosure include an alternating micropattern of adhesive and environmental stimuli-responsive repulsive domains. Thus, the polymer structures in the adhesive domains may differ (in type, composition, conformation, etc.) from the polymer structures making up the environmental stimuli-responsive repulsive domains. In embodiments, the adhesive domains have one or more first polymer structures and are configured to have affinity for a target soft colloid particle. In embodiments, the repulsive domains include one or more second polymer structures that change form a retracted conformation to a swollen conformation in response to an environmental stimulus, such that application of the environmental stimulus activates the second polymer structures to the swollen conformation, while not activating the first polymer structures to a swollen confirmation, such that the repulsive domains enlarge with respect to the adhesive domains. In embodiments the environmental stimulus is essentially a change in environmental factors (e.g., temperature, salinity, pH, humidity, etc.), so while an environmental aspect may be present prior to the change, we refer to the change in stimulus herein as the “application of environmental stimulus”. Also, in the present disclosure, the application of the environmental stimulus/change in stimulus that results in the change of the second polymer structures to the swollen conformation is sometimes referred to as the activation environmental stimulus (in contrast to the environmental conditions or environmental change in stimulus that results in maintenance or reversion to the retracted state).

In embodiments the first polymer structures are made of a polymer that is not responsive to the environmental stimulus that activates the second polymer structures. In other embodiments, the polymer of the first polymer structures may be responsive to a different environmental stimulus or the same environmental stimulus as the second polymer structures, but the activation changes the conformation of the first polymer structures in a different way than the second polymer brushes, such that the effect is still that the repulsive domains enlarge with respect to the adhesive domains.

In embodiments the polymer second polymer structures can be polymer brushes, and the first polymer structures can also be polymer brushes of a different polymer type or they can be a different type of polymer structures such as films, multilayers, networks (e.g., cross-linked polymers), hydrogels and the like. In embodiments, the polymer structures can be grown in situ, grafted, anchored, or otherwise attached to a substrate or other surface.

The affinity of the adhesive domains for the target soft colloid (SC) particle can be designed to be based on one or more affinity factors, such as, but not limited to binding affinity, size affinity, and confirmation affinity. As used herein, affinity includes the preferable attraction and/or retention of a type of particle over one or more other types of particles. For instance, binding affinity would be an affinity factor driven by the attraction and/or retention of corresponding binding motifs on the first polymer structures of the adhesive domains and on the surface of target SC particles. Thus, in embodiments, the first polymer structures can include functional motifs complementary to and capable of reversibly binding complementary motifs on the target SC particle. The binding affinity can be either specific or semi-specific. For instance, the functional motifs on the first polymer structures may be specific for binding motifs found only on the target SC particle or may be semi-specific, in that they bind complementary motifs found on the target SC particle, but also found to some extent on other, non-target particles. In embodiments, the target SC particles may have more of the complementary motifs and/or have a more specific/stronger interaction with the functional motifs on the first polymer structures than non-target cells, such that functional motifs on the first polymer structures have a stronger binding affinity for target SC particles than non-target particles. In embodiments, the first polymer structures in the adhesive domains may have one or more types of functional motifs capable of reversibly binding a complementary motif on a target SC particle, where each type may have a different binding affinity, with some motifs having a stronger binding affinity than others.

In embodiments, the affinity factor can include size affinity, which relates to an affinity of a target SC particle to the adhesive domain based on the size of the domain, size of the particle, or both. Size affinity may be related to the dimensions of the adhesive domain (such as surface area, depth, length/diameter) with respect to the dimensions of the target SC particle. For instance, a small adhesive domain would have a lower affinity for a target SC particle that had larger dimensions than the adhesive domain than an adhesive domain that was large enough to accommodate a greater portion of the target SC particle. The affinity factor can also include conformational affinity, which relates to an affinity of a target SC particle to the adhesive domain based on the deformability/flexibility of the target SC particle (e.g., as compared to non-target particles) and/or the flexibility/conformability of the polymer structure of the adhesive domains. For instance, a more deformable target SC particle may have a greater adhesion energy due to a greater contact area than a non-conformable SC particle. It will also be appreciated and is within the scope of the present disclosure that the adhesive domains can include a combination of affinity factors to establish a greater attraction and/or retention for a target SC particle than a non-target SC particle.

In embodiments the first polymer structures include functional motifs such as described above to provide a binding affinity for a target SC particle. In embodiments the functional motifs on the first polymer structures are complementary to and capable of reversibly binding complementary motifs on the target SC particle. In embodiments, such as when the target SC particle is a rare cell, such as, but not limited to a circulating tumor cell (CTC) (which typically include increased expression of integrins that bind to RGD (Arg-Gly-Asp)—motifs) the functional motif on the first polymer structures may be an RGD motif. While cells other than CTCs and some proteins may also have RGD binding integrins, they typically have fewer such integrins, so the attraction, coupling, and retention (and hence the affinity) to RGD motifs is weaker. Thus, affinity of other, non-target cells and proteins for the RGD motifs on the adhesive domains will not be as strong as the affinity of target circulating tumor cell for the RGD motifs.

In embodiments the target SC particle is a biological SC particle such as, but not limited to, lipid vesicles, living cells, cellular organelles, protein complexes, capsules and the like. In embodiments, the target biological SC particle is a living cell, such as a mammalian cell, such as a human cell. In embodiments, the cell is a rare cell, such as a rare cancer cell (e.g., circulating tumor cell), stem cell, and the like. In embodiments where the target SC particle is a biological particle, the first and second polymer structures are made of biocompatible polymers.

In embodiments, the first polymer structures are made of a polymer that is not responsive to the environmental stimulus that activates the second polymer structures. In embodiments possible non-responsive polymer brushes are made of polymers such as, but not limited to, poly (ethylene oxide), poly(vinyl alcohol), polystyrene, polyacrylates, poly[poly(ethylene glycol) methacrylate] (PEGMA), polysaccharides, polypeptides, polysiloxanes, SU-8 photoresist (commercial product including mixture of Bisphenol A Novolac epoxy with triarylsulfonium/hexafluoroantimonate salt), and others, and copolymers thereof and combinations thereof.

In embodiments, the first polymer structures are polymer brushes that include poly(acrylic acid) (PAA). In embodiments, the PAA polymer brushes can be attached, grafted, etc. to a substrate to form the adhesive domains, such as described in Example 1, below. In other embodiments, the first polymer structure is made of SU-8 photoresist and can be grown directly on a substrate using a mask and standard lithography, such as described in Example 2, below.

In embodiments, the adhesive and repulsive domains can be made of covalently grafted (or strongly anchored/coupled by physical forces) polymer films, polymer networks, and/or polymer hydrogels. In embodiments, the adhesive, non-responsive domains are functionalized to include motifs attractive to (e.g., capable of interacting with) soft colloid motifs, while repulsive domains are made of environmentally responsive (e.g., thermo-, pH-, salt-, electric- and magnetic field, mechanical deformation—responsive) grafted polymers, polymer networks and polymer hydrogels.

As described above, the environmental stimuli-responsive repulsive domains have second polymer structures that are distinct from the first polymer structures, and are made of a polymer that responds to an environmental stimulus, such that the second polymer structures change from a retracted conformation to a swollen conformation in response to an environmental stimulus. Upon activation of the second polymer structures with the application of the environmental stimulus, the second polymer structures change to the swollen conformation which makes the repulsive domains enlarge with respect to the adhesive domains. In this way, the repulsive domains can physically contact and exert a mechanical pushing force on SC particles that may be bound, coupled to, or otherwise physically associated with the adhesive domains. If the pushing force of the repulsive domains is sufficient to overcome the affinity (e.g., binding, coupling, association) of the SC particle for the adhesive domains, it will release, bump, or detach the SC particle from the adhesive domain. In some instances, the enlarging of the repulsive domains may merely “bump” nearby non-target SC particles out of the way to make room for interaction with less numerous target SC particles. In other instances, the activation of the repulsive domain may push away a weakly bound non-target SC particle. In other embodiments, activation of the repulsive domain is strong enough to forcefully detach a bound target SC particle, such as when harvesting cultured cells.

In embodiments the environmental stimulus is temperature, pH, ionic strength, salinity, chemical concentration, electrical field, magnetic field, light (e.g., visual and/or UV light), ligand-protein interactions, mechanical forces (e.g., deformation forces), and the like. In embodiments, the environmental stimulus is a change in one of the above environmental factors from a first status to a second status. For instance, in the case of temperature, the environmental stimulus can be a change from a higher temperature to a lower temperature, or vice versa. In embodiments, it may be a change from an acidic pH to a neutral or basic pH, or vice versa; from a saline environment to a non-saline environment, from a higher calcium concentration to a lower calcium concentration, and the like. In other embodiments, the environmental stimulus may be the application of a heretofore absent stimulation, such as the addition of an electric and/or magnetic field, or mechanical deformation. The environmental stimulus to be applied is tailored to a dynamic polymer that responds by changing conformation in response to application of the stimulus. It will be understood that in order for the repulsive domain to change conformation and enlarge with respect to the adhesive domains, the polymer of the first polymer structure in the adhesive domains should not have the same response to the same environmental stimulus. It may have an opposite response, but typically it will not respond to the environmental stimulus used to activate the second polymer structures of the adhesive domain.

In embodiments, the adhesive domains have a smaller surface area than the target SC particles, such that a target SC particle adhered to an adhesive domain will physically contact a repulsive domain when the repulsive domain swells in response to the environmental stimulus. In embodiments, the repulsive domains have a substantially similar or lower surface height than the adhesive domains in the absence of the environmental stimulus and a greater surface height than the adhesive domains in the presence of the environmental stimulus. Thus, in such embodiments the activation and swelling of the second polymer structures causes the repulsive domains to grow in height, such that the surface height is sufficiently greater than the surface height of the adhesive domains to exert a mechanical “pushing” force on any particles above contacted with a surface of the adhesive domains. In some embodiments the repulsive domains have a dimension ranging from about 5 to 90% of the size of target SC particles (e.g., about 20%-90%).

Typically, a polymer is chosen for the second polymer structures such that the activation level of the environmental stimulus should not be such that it will damage the target SC particle. For instance, in an embodiment where the second polymer structures include a temperature sensitive polymer that changes conformation in response to a change in environmental temperature, generally, the activation temperature should not be a temperature that is damaging to the target particle. In embodiments, if the target SC particle is a biological SC particle (e.g., a cell, protein, lipid vesicle), the activation temperature is not higher or lower than the temperature range for biological viability of the particle.

In embodiments, the environmental stimulus is a change in temperature from a higher to a lower temperature, where the range is within biological temperature ranges. In embodiments, the second thermoresponsive polymer brushes include polymers such as, but not limited to poly(N-isopropylacrylamide) (PNIPAM) and/or other polymers with lower critical solution temperature (LCST). Other stimuli-responsive polymers that can be used for the second polymer structures in the repulsive domains include, but are not limited to, pH-responsive polymers including weak polyelectrolytes, salt responsive polymers composed of weak or strong polyelectrolytes, photoresponsive polymers composed from monomers that undergo photoisomerization, and the like. Polymers responsive to magnetic fields can also be used to accommodate superparamagnetic nanoparticles.

In embodiments, the dynamic polymer surface of the present disclosure can also include an anchoring layer between the micropatterns of adhesive domains and a substrate surface. Substrate surfaces include, silicon wafers, glass slides, polystyrene cell culture dishes, micro- and nanofibers made of synthetic and biopolymers, and the like. In embodiments the anchoring layer is a polymer with epoxy-functional groups, such as but not limited to poly(glycidyl methacrylate) (PGMA), silanes with epoxy functional groups, silanes with amino functional groups, cross-linkable copolymers that contain major functional groups such as hydroxyl, carboxyl, epoxy, amino, azide, alkyne, cycloalkyne, alkene, isocyanate, photoresist materials (e.g., SU-8), and the like. In some embodiments, these polymers, such as SU-8 can be used to form the first polymer structures and can be functionalized with motifs having an affinity for the target SC particles.

In embodiments, the alternating micropattern of adhesive domains and environmental stimuli-responsive repulsive domains can have a regular, structured pattern (such as a grid-like pattern, or other defined, repeating pattern). In other embodiments, the alternating micropattern of adhesive domains and environmental stimuli-responsive repulsive domains can have an irregular and/or randomly scattered patterns. As used herein “alternating micropattern” does not require that the adhesive and repulsive domains have a 1-to-1 alternating pattern, but can be a more irregular pattern of adhesive and repulsive domains. The regularity or randomness/irregularity of the pattern can be a function of the method of synthesizing the dynamic polymer surface, as explained in greater detail below. For instance, if photolithography techniques are used, a regular, structured pattern of adhesive and repulsive domains can be produced, whereas methods such as phase-separation and dewetting produce a more irregular and randomly distributed patterns of adhesive and repulsive domains.

In some embodiments, the dynamic polymer surface of the present disclosure may include a third domain, which is a subset of the environmental stimuli-responsive repulsive domains. This may be useful when two different levels of repulsion are desired or a response to two different stimuli are needed, or both. For instance, when scaling up cell culture methods, most of the particles in the environment will be target cells, most will be adhered to the adhesive domains, and a single “push” may be sufficient for releasing the cells for harvesting. However, in some embodiments, many cells or other particles may be present in the environment, and target cells or particles may be rare, such as in screening and enrichment of circulating tumor cells. In such applications it may be desirable to have two or more levels of repulsion. For instance, the surface can include a weaker “push” from some repulsive domains to bump away and detach nearby or weakly bound non-target particles and/or cells; however, a stronger “push” may be needed, such as from a second repulsive domain, to detach and release bound target particles and/or cells. In such situations, the second repulsive domain can have a stronger “push” and typically can be configured to be activated in response to a different environmental stimulus so as not to detach a substantial amount of bound target particles/cells in every activation of the primary stimuli-responsive repulsive domains. Thus, in some embodiments, a portion of the environmental stimuli-responsive repulsive domains are activatable super-repulsive domains. The activatable super-repulsive domains include one or more third polymer structures (e.g., polymer brushes and other structures) that change conformation in response to a second environmental stimulus such that, upon application of the second environmental stimulus, the activatable super-repulsive domains swell and enlarge with respect to the adhesive domains as well as the environmental stimuli-responsive repulsive domains comprising the second polymer brushes. The activated/enlarged super-repulsive domains will typically have a greater surface dimension/height than the adhesive domains and the activated second polymer structures of the environmental stimuli-responsive repulsive domains. In embodiments, the second environmental stimulus is different than the environmental stimulus that activates the second polymer structures such that the second polymer structures and third polymer structures are not activated simultaneously unless both environmental stimuli are applied.

The present disclosure also includes products/substrates coated with the dynamic polymer surface of the present disclosure as described above. In embodiments, the products also include a controlled environment to house the substrate with the dynamic polymer surface coating. The controlled environment allows a user to control/modulate the environment in order to affect the response of the environmentally responsive repulsive domains. In embodiments, the product is a cell culture system, such as substrates coated with the dynamic polymer surface for culturing cells, and a controlled environment for culturing the cells (e.g., a housing/container, culture media, incubator for temperature controls, etc.). In embodiments, a cell culture system of the present disclosure further includes instructions for growing target cells in the system and for application of the environmental stimuli for release and harvesting of the cells. In some embodiments, the product is a screening system for selecting and enriching target soft colloid particles and further includes instructions for changing the environmental stimuli for adhesion or repulsion of target soft colloid particles.

Methods of Making Dynamic Polymer Surfaces

Embodiments of the present disclosure further include methods of making dynamic polymer surfaces of the present disclosure. In embodiments, methods of making a dynamic polymer surface of the present disclosure include providing a solid substrate having a surface and forming a dynamic polymer layer of the present disclosure on the substrate surface.

In some embodiments, the methods of the present disclosure include optionally modifying the surface of the solid substrate with an anchoring polymer layer on which the dynamic polymer layer can be formed. The methods of the present disclosure further include forming a dynamic polymer layer directly on the substrate surface or on the anchoring polymer layer, the dynamic polymer layer having an alternating micropattern of adhesive domains and environmental stimuli-responsive repulsive domains, where the adhesive domains include one or more first polymer structures configured to have affinity for a target soft colloid particle, and the repulsive domains include one or more second polymer structures that change conformation in response to an environmental stimulus such that application of the environmental stimulus activates the second polymer structures, but does not activate the first polymer structures (or produces a different reaction) such that the repulsive domains swell and enlarge with respect to the adhesive domains.

In embodiments of the methods of the present disclosure, the alternating micropattern of adhesive domains and environmental stimuli-responsive repulsive domains is a regular, structured pattern and the polymer layer is formed using photolithography methods. In embodiments, the methods of forming the regular structured micropattern of adhesive and response domains includes preparing an anchoring polymer layer and then functionalizing the anchoring polymer layer with photoresist and using a photomask with a structured micropattern to define the alternating domains by protecting the photoresist areas of the substrate covered by the photomask while deprotecting areas not protected by the photomask. Then the first polymer structures or second polymer structures are grown on the deprotected areas to form the adhesive domains or the environmental stimuli-responsive repulsive domains. The method can also include deprotecting the photoresist in the areas previously protected by the photomask and growing the other of the first polymer structures or second polymer structures on the deprotected areas previously protected by the photomask to form the other of the adhesive domains or environmental stimuli-responsive repulsive domains. Variations of this method can be used to provide the polymer layer having an alternating micropattern of adhesive domains and environmental stimuli-responsive repulsive domains. Additional details regarding embodiments of such methods are provided in the examples below.

In embodiments of the photolithography methods described above, such as in the embodiment described in detail in Example 1, a polymer anchoring layer can be formed that includes a polymer with epoxy-functionalized groups, such as, but not limited to, poly(glycidyl methacrylate) (PGMA). In some embodiments, the photoresists is cyclopropenone-caged dibenzocyclooctyne-amine (photo-DIBO). In embodiments, such as in Example 1, the photo-DIBO photoresist was attached to the PGMA anchoring layer, a mask was used and UV light was applied to define the alternating adhesive and repulsive domains. After deprotection, the irradiated portions were functionalized with azido-BIBB and grafted with PNIPAM brushes. The grafted PNIPAM was protected to prevent initiation of further polymerization and the surface was again exposed to UV source to activate residual photo-DIBO that was previously masked/protected. In these areas, PAA polymer brushes were formed (e.g., by polymerization of PtBA brushes and then hydrolyzing the PtBA brushes into PAA brushes). The PAA brushes were grafted to conjugate RGD peptide through EDC/NHS chemistry to provide binding affinity motifs to form PAA-RGD adhesive domains.

In other embodiments, such as described in greater detail in Example 2, SU-8 photoresist was used to provide the microdomain pattern. The substrate surface was functionalized with APTES and BIBB. SU-8 photoresist was then coated on the functionalized surface, a photomask was applied, and SU-8 polymer structures were formed in the irradiated (unmasked) areas to form the adhesive domains. NIPAM monomers were then polymerized on the previously masked portions to form temperature responsive PNIPAM polymer brushes representing the repulsive domains. RGD adhesive motifs were conjugated on the surface of the SU-8 polymer surfaces to define the adhesive domains. The RGD motifs can be conjugated directly on the surface of SU-8 through amine-epoxy reaction without grafting additional polymer brushes in the adhesive domain (as with the PAA brushes in the other embodiment). This method of making involved fewer steps and did not require the use of DIBO which is not widely commercially available.

In other embodiments of making the dynamic polymer surfaces of the present disclosure, methods other than photolithography can be used that are amendable to larger scale production. With some such methods, the alternating micropattern of adhesive domains and environmental stimuli-responsive repulsive domains has a somewhat irregular pattern (e.g. as compared to the structured/regular pattern formed with the photolithography methods described above). One such method includes phase-separation and includes first dissolving a first polymer and a second polymer in a common solvent to form a mixed polymer solvent composition, where the first polymer and second polymer are immiscible. The methods then include casting a film of the mixed polymer solvent composition on the surface of the substrate. Then the solvent is evaporated to form the polymer layer having an alternating micropattern of adhesive domains and environmental stimuli-responsive repulsive domains due to the phase separation of two incompatible polymers in the film.

In other embodiments, the dynamic polymer surface is formed using dewetting methods and also produces an alternating micropattern of adhesive domains and environmental stimuli-responsive repulsive domains having an irregular pattern (as compared to the photolithography methods). In embodiments, the dewetting methods include dissolving a first polymer for forming adhesive polymer film in a first solvent to form a first polymer solution and dissolving a second polymer for forming repulsive domains in a second solvent to form a second polymer solution, where the solvent for the second polymer is nonsolvent for the first polymer. In embodiments, dewetting methods then include casting a film of the first polymer solution on the surface of the substrate and evaporating the first solvent to form a first polymer film, followed by casting a film of the second polymer solution on the first polymer film, such that the second polymer solution dewets and forms micro-patterns upon solvent evaporation that results in the formation of alternating micropatterns of adhesive domains and environmental stimuli-responsive repulsive domains. Additional details regarding the phase separation and dewetting methods are described in the Examples below, and a skilled artisan will understand variations of such methods can be employed according to methods of the present disclosure.

Methods of Enriching and/or Culturing Cells Using Dynamic Polymer Surfaces

The present disclosure also includes methods of using the dynamic polymer surfaces, and products including the dynamic polymer surfaces for applications such as enrichment of target particles in a sample, cell culturing and harvesting, and the like. Methods include non-enzymatically detaching particles from a polymer surface using the dynamic polymer surfaces of the present disclosure described above. These methods of detachment can be used in cell culturing and harvesting applications as well as in cell sorting/enriching applications.

Methods of enriching target particles in a sample and cell culture and harvesting methods involve the step of detaching particles (e.g., target particles, such as target cells, etc.) from a surface, preferably using non-enzymatic approaches due to the disadvantages of enzymatic approaches discussed above. Thus, methods of the present disclosure include methods for non-enzymatically detaching particles from a polymer surface. In embodiments, such methods can include contacting the dynamic polymer surface of the present disclosure discussed above with a liquid composition comprising particles with affinity for the adhesive domains in a controlled environment the absence of the environmental stimulus for a first period of time during which the repulsive domains are in a retracted conformation. During this time, particles with an affinity for the adhesive domains can attach to/become associated with the adhesive domains. Such methods can then include applying the environmental stimulus to the controlled environment for a second period of time to activate the second polymer structures to the swollen conformation such that the repulsive domains enlarge with respect to the adhesive domains, thereby physically contacting and exerting a mechanical force on particles adhered to the adhesive domains sufficient to detach a portion of particles from the adhesive domains.

In some embodiments, the liquid composition includes target soft colloid particles and one or more other types of particles (non-target particles) and the method includes separating the target soft colloid particles from the non-target particles in the liquid composition (e.g., liquid sample). In some such embodiments, at least some of the non-target particles can have a mild, non-specific affinity for the adhesive domains, and thus may have a weak interaction/binding with the adhesive domains. However, due to the design of the adhesive domains (as discussed above), the target soft colloidal particles have a specific affinity for the adhesive domains that is stronger than the affinity of the non-target particle, so that the target soft colloid particles (SCPs) have a stronger attachment/interaction with the adhesive domains than the non-target particles. The method then includes applying one or more of the environmental stimuli discussed above to the controlled environment for a second period of time to activate the second polymer structures in the repulsive domains. The environmental stimulus activates the second polymer structures to the swollen conformation such that the repulsive domains enlarge with respect to the adhesive domains. Due to the difference in strength/affinity of attraction/binding between target SCPs and the adhesive domains versus the weaker affinity of non-target particles, applying the environmental stimulus to the controlled environment for the second period of time to activate the second polymer structures to the swollen conformation is effective to detach a majority of the non-target particles from the adhesive domains while retaining target soft colloid particles with a stronger affinity adhered to the adhesive domains.

In embodiments, methods further include removing the environmental stimulus for a third period of time, such that the repulsive domains return to the retracted confirmation. Embodiments of methods can include repeating the application and then removal of the environmental stimulus for a number of cycles effective to increase the percent of target soft colloid particles bound to the adhesive domains. In effect, the repeated cycles continue to deflect non-target particles with each repulsive cycle (application of environmental stimuli), while capturing more target particles with each adhesive cycle (removal of environmental stimuli) until a greater percentage of target SCPs are attached to adhesive domains. This can enrich the number of bound SCPs from a sample, even if the number of target SCPs in a sample is very low (as in the case or rare SCPs, such as circulating tumor cells in a sample from a subject).

After a number of cycles, the method can include removing the liquid composition from the controlled environment to retain bound target soft colloid particles on the dynamic polymer surface. In embodiments, after removing the liquid composition with non-bound particles, the bound target SCPs can be released releasing bound target soft colloid particles from the adhesive domains to obtain an enriched sample of target soft colloid particles. In embodiments, the bound target SCPs can be released by other methods discussed above. The environmental stimulus can be any of the environmental stimuli (or combinations thereof) as discussed above, depending on the polymer used for the adhesive domains. In some embodiments, the environmental stimulus is a change in environmental temperature from a first temperature to a second temperature, wherein neither the first temperature nor second temperature damages target particles. In embodiments, the change in temperature is a reduction in temperature.

In some embodiments of the methods above, the dynamic polymer surface includes a portion of the environmental stimuli-responsive domains that are activatable super-repulsive domains such as described above having one or more third polymer structures that change conformation in response to a second environmental stimulus (different from the first environmental stimulus) such that, upon application of the second environmental stimulus, the activatable super-repulsive domains swell and enlarge with respect to the adhesive domains and the environmental stimuli-responsive repulsive domains comprising the second polymer structures and have a greater surface height than the adhesive domains and the environmental stimuli-responsive repulsive domains comprising the second polymer structures. In such embodiments, releasing bound target soft colloid particles from the adhesive domains includes applying the second environmental stimulus to the controlled environment such that the activatable super-repulsive domains swell and enlarge effective to detach bound soft colloid particles from the adhesive domains. In embodiments of the methods of the present disclosure, the environmental stimulus can be a change in environmental temperature from a first temperature to a second temperature, and the second environmental stimulus can be, but is not limited to, a change in environmental pH, a change in environmental salinity, a change in environmental chemical concentration, a change in temperature to a third temperature, or a combination thereof.

In some embodiments of the methods of the present disclosure, the liquid composition can be a biological sample and the target soft colloid particles can be, but are not limited to, lipid vesicles, cells, cellular organelles, protein clusters and complexes, polymer capsules, and microgel particles. In embodiments, the target soft colloid particles are rare cells, and the one or more other types of particles comprise other cells. In some such embodiments, the biological sample can be selected from the group including, but not limited to, whole blood, plasma, urine, saliva, biological secretions, biopsy samples, tissue samples in a biocompatible medium, in vitro expanded cells, and combinations thereof. In embodiments, the rare cells are circulating tumor cells (CTCs).

Embodiments of the present disclosure also include methods of for culturing/growing and harvesting target cells. In embodiments, the particles include a plurality of target cells with an affinity for the adhesive domains, and the liquid composition can include a growth medium effective to grow and proliferate the plurality of target cells. In such methods, the first period of time is an amount of time effective to increase the number of target cells to a desired amount. After the cells have reached a desired confluency, then the methods can include applying the environmental stimulus to the controlled environment for a second period of time effective to detach target cells from the adhesive domains. In embodiments, after the cells are detached, the methods can further include harvesting the detached target cells from the controlled environment. In embodiments of such methods, the number of target cells can be increased by about 25% or more, about 50% or more, about 100% or more, and the like. In embodiments of the methods of the present disclosure at least a majority of harvested target cells are viable and/or undamaged. These methods of the present disclosure can be used with products such as described above including the dynamic polymer surfaces for scaling up growth and harvesting of cells for various purposes as discussed above.

Additional details regarding the compositions, systems, and methods of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1—Thermo-Responsive Switchable Interfaces with PNIPAM and PtBA Domains for Isolation & Enrichment of Mammalian Cells

The immune system response, which relies on labeling pathogens with antibodies to destroy invaders, is an example of how live organisms discriminate certain particulates in a complex colloidal mixture in a blood flow. This labeling concept was successfully mimicked in vitro for analytical, diagnostic, and research applications; however, it became prohibitively expensive for mass production and also when the use of a gene-editing mechanism for labeling is not feasible. Scalable separation methods such as gas- and liquid chromatography explore a series of adsorption columns when the separation is approached through rapid multiple adsorption-desorption steps of the dynamic equilibrium state in a mixture of molecules with different partition coefficients. Such a scalable sorting and purification of colloidal particulates, including protein complexes, cells, and viruses, is limited due to a high energy barrier, up to millions kT, required to detach particles from the interface, which is in dramatic contrast to a few kT observed for small molecules. Such a strong interaction renders particle adsorption quasi-irreversible. The dynamic adsorption-desorption equilibrium is approached very slowly, if ever attainable. The dynamic polymer structures of the present disclosure overcame this limitation with a local oscillating repulsive mechanical force generated at the microstructured stimuli-responsive polymer interface to switch between adsorption and mechanical-force-facilitated desorption of the particles. The switching occurs at a low frequency to push the particle out and to match a slow transport of particles dragged by a solution flow. Such a dynamic regime enables the separation of colloidal mixtures based on the particle-polymer interface affinity. This design of the interface could find use in research, diagnostics, and industrial-scale label-free sorting of cells and other kinds of particles, for example, isolation of one in billions of cells based on the expression of their membrane receptors as well as in cell culture and harvesting applications.

### Introduction

Synthesis of molecules, fine particles, and expansion of live cells involve statistical chemical and physical interactions, side reactions, mutations, and mixing with pathogens (for live cell systems in the latter two cases), resulting in polydisperse materials by dimensions and functionality that require discrimination, purification, and sorting.

Scalable sorting is challenging for fine particulates—materials with dimensions greater than 1 nm and smaller than 100 μm. This range is typically associated with colloidal particles, including nanoparticles, protein complexes, live and dead cells, unicellular organisms, and viruses. Among existing highly selective sorting methods for cells and other colloidal particles are the Coulter method,¹ later extended to flow cytometry,² antibodies-decorated magnetic beads,³ and microfluidic methods,⁴ including methods with the aid of magnetic particles,^{5, 6} surface acoustic waves,⁶ and dielectrophoresis.⁷ Although existing methods are highly accurate, they indeed are limited to analytical, diagnostic, and research applications dealing with a relatively small volume of cells or colloids. The need for laboratory and industrial scaled-up cell sorting in extensive studies and mass production is rapidly increasing with rising numbers of new technologies that rely on sizable cell expansion for plentiful applications from therapeutic cells to in vitro grown artificial meat.

Separation and sorting based on the physical properties of substances are major processes used for analysis, diagnostics, and manufacturing technologies. Unlike macroscopic objects that are sorted based on their size, shape, density, weight, and mechanical properties, the separation and sorting of fine particles can be different. Phase separation and centrifugation, distillation, chromatography, and membrane filtration are major processes for scalable separation, sorting, and purification of molecules. These separation methods find limited applications for particles of submicron and micron dimensions. Several factors cause these limitations: (i) colloidal particles are typically dispersed in media and cannot exist in a dry form because of a strong tendency to aggregation and, in the case of live cells, possible death; (ii) due to a larger size as compared with molecules, colloids diffuse slowly; (iii) for many materials, variations in materials density and dimensions are minimal. For these materials, a separation based on affinity to adsorbents would be more efficient. However, colloids become entrapped at interfaces when the thermal energy of colloids is below the desorption activation barrier due to a high particle—adsorbent or particle—liquid interface contact area per particle.

Large molecules can be efficiently sorted at industrial scales by their affinity to adsorbents with gas- or liquid chromatography methods based on competitive adsorption-desorption steps rapidly approaching dynamic equilibrium. Liquid chromatography is problematic for high molecular weight polar polymers and much less efficient for particulates because of the quasi-irreversible adsorption of large objects with multisite adsorbent-adsorbate interactions. The interaction is not approaching the equilibrium state because of slow desorption and slow surface diffusion, which hinders the affinity-guided competition for adsorption sites at the interface.⁸⁻¹⁰ The free energy required to remove micrometer particles of radius R entrapped at the interface is proportional to R², and it reaches 105-10⁸ kT in striking contrast to just several kT for small molecules.¹¹

An affinity-based sorting of colloids could be achieved by overcoming the high desorption energy barrier using external sources of energy, for example, shear flow¹² or ultrasound.^{13, 14} However, for the affinity-based separation, the detachment or desorption forces should be balanced by adhesive or adsorption forces to provide a dynamic attachment-detachment process that resembles dynamic adsorption-desorption of small molecules at interfaces driven by thermal fluctuations. Such dynamic equilibrium can be established when the “desorption hysteresis” is minimized by precise tuning of oscillating repulsive force with a period matching a slow transport of the colloids in the medium to support mechanisms for the competitive adsorption-desorption process and transport-driven exchange between particles at the interface and in bulk. For example, antibody-antigen interactions are highly selective but very strong. This type of binding is not easy to break down. Consequently, the equilibrium state in a case of multiple antibody-antigen interactions per particle (a typical case of functional particles and live cells) is difficult to approach if ever possible.

A precise and uniform adjustment of detachment forces using liquid flux or ultrasound sources is difficult to approach at large scales. Using a shear force for cell detachment in microfluidic devices has limitations for scaling up because the drag force depends on the radius-to-length ratio of the liquid flow cell at the given pressure drop. An increase in the dimension of the flow cell is practically limited by a possible increase in pressure. It is also difficult to approach a uniform propagation of ultrasound waves in sizeable setups due to attenuation in a viscoelastic medium.

The present example describes the development of a cell detachment solution using stimuli-responsive materials. Thermoresponsive polymer interfaces were fabricated using tethered polymers—a polymer brush architecture constituted of two distinct adhesive and repulsive domains of the microstructured material when the local repulsive behavior of the interface is due to a mechanical force applied to adsorbed particles. The force is oscillating between minimal and maximal values by periodic variations of temperature.

Of note is the difference between the well-known phenomenon of adsorption and adhesion alternation with thermoresponsive polymer systems and the proposed approach here. Changes in adhesion in typical thermoresponsive systems are based on a shift in the polymer-solvent-adsorbate interaction equilibrium toward preferential either polymer solvent or polymer adsorbate interactions when the same surface alternates from non-adhesive to adhesive behavior.¹⁵ Such a shift is typically moderate (with a factor 2-3) in terms of the adhesion energy scale.¹⁶ Consequently, a possible separation is based on a low affinity level.

This example describes the interface when adhesive and repulsive behavior is spatially divided. The interface is constituted of two types of microdomains with (i) permanently adhesive and (ii) oscillating repulsive behavior. The level of adhesive interactions can be as broad as those from nonspecific weak interactions to very strong antibody-antigen interactions. The amplitude of the oscillating repulsive force varies from 0 to 10⁵ N/m². This combination enables separation based on a wide range of affinity interactions adjusted to the specific colloidal system. The concept is proved in a series of experiments with different mammalian cells.

### Materials and Methods

Materials. Si-wafers were purchased from University Wafer, Boston, Mass., USA. Photo-DIBO-amine was synthesized as previously described.²³ Poly(glycidyl methacrylate) (PGMA, Mn=20 kg/mol), ascorbic acid (ASCO), 2-azidoethyl 2-bromoisobutyrate (azido-bibb), copper(II) bromide (CuBr₂), N,N,N′,N″,N″-pentamethyldiethylenetriamine (PMDTA), N-isopropylacrylamide (97%) (NIPAM), tert-butyl acrylate (98%) (tBA), dimethylformamide (DMF), ammonium hydroxide solution 28% NH₃ in H₂O (NH₃OH), 30% hydrogen peroxide, RGD peptide, 1-ethyl-3-(3-dimethylaminopropyl)carbodiimide (EDC), and N-hydroxysuccinimide (NHS) were purchased from Sigma and used as received. NIPAM was used after recrystallization from hexane, and tBA was used after purification by filtering through the neutral aluminum oxide to remove inhibitor prior to the polymerization. Glider TEM grids were purchased from TED PELLA, INC. For cell experiments, Dulbecco's modified eagle medium (DMEM), wheat germ agglutinin (WGA), and Vybrant™ DID were received from Thermofisher Scientific. U-87MG cells, a human glioblastoma cancer cell line, and red fluorescence protein transfected B16F10 (RFP-B16F10), a murine melanoma cancer cell line, were received from Dr. Jin Xie lab.

Measurement. Atomic Force Microscopy (AFM) images were obtained using the Bruker Multimode Nanoscope MM8 in tapping mode. Brush thickness was estimated with a Nanofilm ep4 imaging null-ellipsometer, Accurion Inc. (Germany). A fluorescence microscope (Olympus BX51) was used to monitor the isolation of fluorescently labeled cells. Hemacytometer and Nageotte counting chamber (Hausser Scientific, Horsham, Pa.) were used to determine the concentration of cells. Molecular mass of polymers was measured using a house-made membrane osmometer, and gel permeation chromatography (GPC), a Shimadzu LC-20 Ad series equipped with a RID-10A refractive index detector and the chloroform was used as mobile phase.

### Preparation of Samples.

Functionalization of Silicon Wafers with DIBO. The 1×1 cm² cut samples of the Si-wafer were sonicated in ethanol for 2 min and rinsed with DI water. The surface of silicon wafers was then cleaned in a solution of 28% NH₃OH/30% H₂O₂/DI water (1:1:1 by volume) for 1 h at 60° C., rinsed with DI water, and dried under a flux of argon gas. The surface of the cleaned samples was functionalized with photo-DIBO as previously described:²⁴ a PGMA solution (0.25% in chloroform) was spin-coated on the wafers and crosslinked by annealing in an oven at 110° C. for 1 h under vacuum. Non-crosslinked PGMA was removed by soaking the samples in hot chloroform for 30 min. Photo-DIBO-amine was covalently bound to the PGMA layer by incubating samples in photo-DIBO-amine solution (12.5 mg/mL in methanol) at 53° C. for 24 h. The samples were rinsed with methanol and dimethylformamide.

Synthesis of patterned PNIPAM brushes on DIBO-modified silicon wafers. A TEM grid was placed on the photo-DIBO-modified samples and covered by a glass slide and exposed for 2.5 min to a UV-source (Spectroline ENF-240C, 365 nm wavelength, 1 mW/cm²). The irradiated sample was functionalized with azido-bibb (5 μL in 100 μL of dry DMF) for 1 h. PNIPAM brush was grafted via ARGET-ATRP as described elsewhere:²² 9 μL of 0.22 M CuBr₂ and 9 μL of 0.48 M PMDTA were added to 40% of NIPAM monomer solution (0.4 g in 300 μL of DI water and 700 μL of methanol). Oxygen was removed by purging the solution with argon gas. Then, 50 μL of ASCO (0.04 g/mL) was added slowly into the sealed polymerization reactor. The polymerization was allowed to proceed for 2 h at ambient temperature. All steps involving photo-DIBO were carried out in a darkroom. The polymerization was stopped by opening the cap, and samples were rinsed with ethanol followed by drying under a flux of argon gas.

Synthesis of PAA brush and conjugation of RGD. The samples with previously grafted PNIPAM were exposed to a sodium azide solution (0.04 g/mL in DI water) for 1 h to transform the bromine end groups of PNIPAM into azide groups to prevent the initiation of further polymerization and formation of a block copolymer. Next, the samples were exposed to the UV-source again to activate the residual photo-DIBO molecules in the previously masked areas. As described above, then the samples were functionalized with the ATRP initiator. For the polymerization of tBA, samples were placed in a polymerization reactor: 9 μL of 0.22 M CuBr₂, 9 μL of 0.48 M PMDTA, 500 μL tBA and 1.5 mL of ethanol. After purging the polymerization reactor with argon gas, 50 μL of ASCO (0.04 g/mL) was added. The reaction proceeded for 20 min at ambient temperature, followed by rinsing the samples with ethanol. PtBA brushes were hydrolyzed into PAA brushes in a 2% of methanesulfonic acid solution in extra dry dichloromethane for 2 min at room temperature. RGD was conjugated through an amidation reaction between the carboxyl groups of PAA and primary amine groups of RGDs in presence of EDC and NHS to activate the acrylic acid groups in MES buffer (pH 6): 20 mg of EDC and 25 mg of NHS were dissolved in 1 mL of MES buffer, and 100 μL of the solution were used for each sample. After 1 h, amine-reactive sulfo-NHS ester was formed, and the sample was rinsed using MES buffer and PBS buffer. Then, the sample was incubated in 100 μL of RGD solution (0.125 mg/mL in PBS buffer, pH 7.4) for 6 h.

NIH/3T3 Cells Attachment and Detachment. DMEM medium that was supplemented with 10% FBS and 100 U/mL penicillin-streptomycin was used for NIH/3T3 cell culture. Cells were expanded under normal conditions (in 37° C. and 5% CO₂ humid chamber). Freshly prepared brush samples were sterilized using a 75% ethanol solution in a 6-well plate and rinsed with a sterile PBS buffer. For cell incubation, 3×10⁵ NIH/3T3 cells were seeded onto a 6-well plate and incubated at 37° C. and 5% CO₂ for 24 h. The cells were stained with calcein-AM for analysis purposes. Cell detachment was achieved by cooling the 6-well plate to room temperature. The samples were taken out and imaged under a fluorescence microscope. Cell numbers were counted using ImageJ.

Capture of Cancer Cells from whole blood. U-87MG cells were grown in DMEM medium containing 10% FBS, 1× non-essential amino acids, and 100 U/mL penicillin-streptomycin and cultured at 37° C. with 5% CO₂ in a humidity incubator. After expansion, U-87MG cancer cells were collected via trypsinization and lightly fixed with formalin for 5 min. Then, cells were washed with cold PBS twice and stained with WGA or DiD for fluorescence imaging purposes. FITC and APC channels were used to image the WGA-stained cells and DID-stained cells, respectively. Healthy human blood samples were used as a non-target cells to suspend the CTCs in the experiment. The human blood treated with the anticoagulant EDTA was obtained from Zen-Bio (Research Triangle Park, N.C.). The impact of cell concentration on CTC isolation efficiency was investigated with a series of spiked samples with the U-87MG to whole blood cells ratio ranging from 1:2×10³ to 1:1×10⁹, mimicking CTCs concentrations in a real patient. U-87MG cells were diluted prior to being added into the blood suspension. The concentration of diluted U-87MG cells was determined by a hemacytometer and Nageotte counting chamber for a lower concentration of cells. Ahead of the isolation experiment, the brush samples were sterilized in 75% of ethanol and rinsed with fresh sterile PBS buffer. The sample was mounted into the liquid flow cell connected to the circulation system. Each sample was incubated for a certain period of time, ranging from 30 min to 6 h in the liquid flow cells with periodically alternating temperature. Afterward, the samples were taken out and analyzed with a fluorescent microscope.

Isolated cell proliferation. The experiments with live RFP-B16F10 murine melanoma cancer cell line was conducted using the same protocol as described for U-87MG cells. After isolation, the brush sample with captured cells was gently washed with PBS buffer and incubated in a 24-well plate with full growth medium under normal conditions for 6 days. During incubation, the medium was replenished every day. At the end of incubation, cells were fixed with formalin for characterization and same area of the sample surface was imaged immediately after isolation and after a 6-day expansion.

### Results and Discussion

Design of Stimuli-Responsive Interfaces with Oscillating Adhesive-Repulsive Forces.

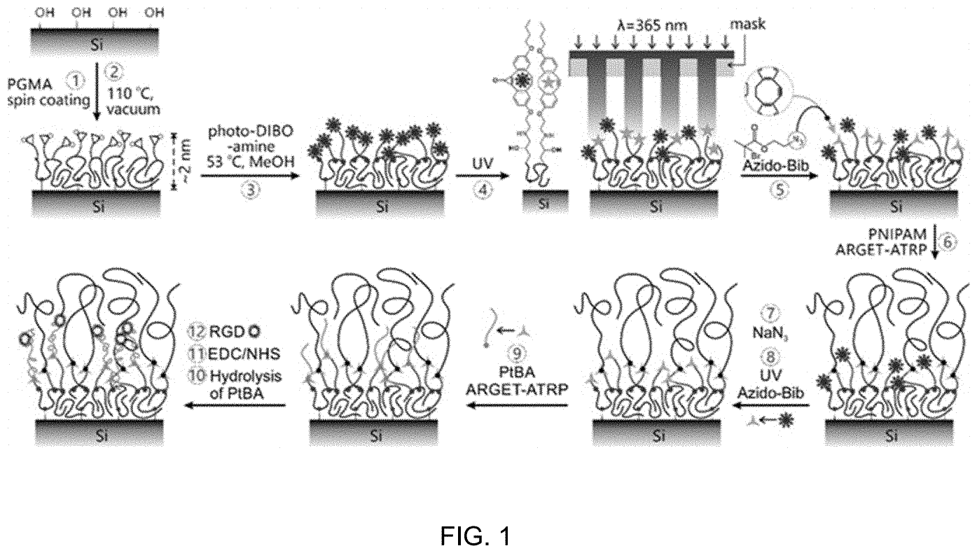

The microstructured binary polymer brushes are composed of poly(N-isopropylacrylamide) (PNIPAM) domains and cell-adhesive tripeptide Arg-Gly-Asp (RGD) conjugated polyacrylic acid (RGD-PAA) domains. RGD is an example of a commonly used adhesive peptide sequence present in extracellular matrix (ECM) proteins that bind to the cell adhesion protein integrin.¹⁷⁻²⁰ Cell adhesion to various interfaces is regulated by the concentration of surface-bound RGD.²¹ A schematic illustration of the microstructured polymer surface is shown in FIG. 1. Briefly, FIG. 1 shows the steps of, PGMA coating (1)/(2), Attachment of photo-DIBO at 53° C. overnight (3), applying photomask and UV light (365 nm) (4), immobilization of Azido-Bib initiator on the irradiated areas (4), formation of PNIPAM repulsive domains (6), activation of previously protected areas (7/8), formation of PAA polymer structures (9-11); and RGD conjugation (12).

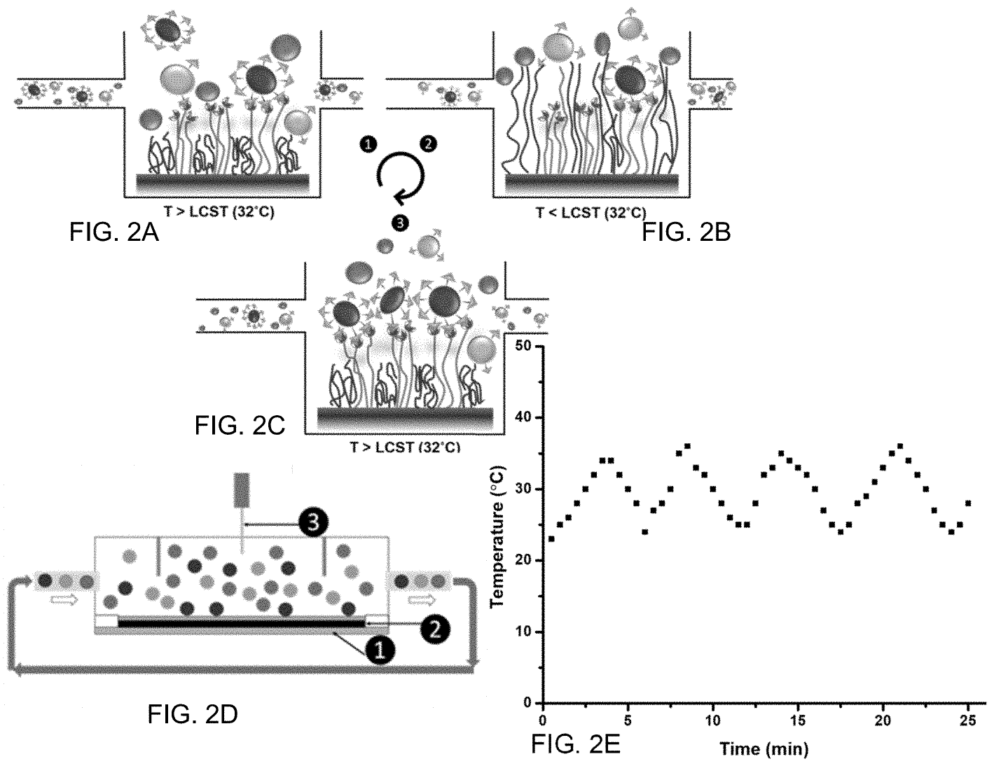

The design concept of the embodiment of the dynamic polymer brush layer is schematically shown in FIGS. 2A-2C. The PNIPAM brush in aqueous media is a thermoresponsive system. PNIPAM is collapsed at a temperature above the lower critical solution temperature (LCST) T_{LCST}=32° C. (FIG. 2A). In contrast, the brush is highly swollen at a temperature below LCST (FIG. 2B). RGD-PAA brush is highly swollen in aqueous media at pH>5. RGD-PAA domains are considered here as cell-adhesive domains. PNIPAM domains are considered as cell-repulsive domains at a temperature below T_{LCST}. Switching from the adhesive state to a strong repulsion is approached if the swollen RGD-PAA brush is substantially thicker than the collapsed PNIPAM brush at a temperature above T_{LCST}, and the swollen PNIPAM brush is substantially thicker than RGD-PAA brush at a temperature below T_{LCST}. Cells are bound to RGD-PAA domains at T>T_{LCST}; however, they are pushed away by the mechanical force of the swollen PNIPAM domains at T<T_{LCST}. Multiple repetitions of the switching between adhesive and repulsive states drive the system to equilibrium affinity-based adsorption of the colloids (FIG. 2C). The diffusion of colloids is slow. The adsorbent carrying the polymer brush is mounted in a liquid flow cell for the improvement of mass transport between the solution and the interface using the drag force (FIG. 2D). The switching between adhesive and repulsive regimes is realized by a periodic alternation of temperature in the cell (FIG. 2E).

This type of stimuli-responsive interface can be prepared on the surface of plane substrates or glass beads for use in small-scale analytical devices and in a large-scale separation column or as a pore wall coating in porous structures permeable for cells and other particles. For the support of this concept, we use microstructured brush-decorated silicon wafers (Si-wafers) mounted on the bottom of a flow liquid cell with the horizontal thermoconductive bottom wall (FIG. 2D) that rests on the surface of a programmable cold/hot plate. In the experiment, we use a close loop flow system to monitor the development of the state of equilibrium adsorption for asymmetric mixtures of cells with different levels of integrin expression. In contrast to one-component PNIPAM thermoresponsive interfaces, when adsorption-desorption changes are due to the change of PNIPM hydrophilicity, the two-component system is universally applied for different affinity mechanisms in a very broad range of adhesive interactions.

Synthesis of microstructured binary polymer brushes. Polymer brushes were grafted to the Si-wafer substrates using an activator regenerated by electron transfer atom transfer radical polymerization (ARGET-ATRP) as described in Ionov, et al., 2021 (which is hereby incorporated by reference herein). The well-controlled polymerizations of PNIPAM and PtBA are supported by the polymer brush thickness (data not shown). The surface of Si-wafers was functionalized with epoxy-functional groups by casting and temperature annealing of a thin poly(glycidyl methacrylate) (PGMA) layer, followed by binding cyclopropenone-caged dibenzocyclooctyneamine (photo-DIBO-amine)²³ as previously reported in Laradji, et al, 2016 (incorporated by reference herein). A 2000 mesh TEM grid with 7.5 μm square hole width and 2.5 μm bar width was used as a photomask to activate the photo-DIBO in the UV-irradiated (365 nm wavelength) areas and to click the 2-azidoethyl 2-bromoisobutyrate (azido-bibb) ATRP initiator. After polymerization of the NIPAM monomer and deactivation of the end Br functionalities of the brush, the sample was UV-irradiated for the second time to activate the previously masked photo-DIBO. The activated and then initiator bound surface was used to graft poly(tert-butyl acrylate) (PtBA) brush. The PtBA brush was, next, hydrolyzed to the poly(acrylic acid) (PAA) brush, followed by conjugation with RGD through the EDC/NHS coupling method. Each step of the synthesis was characterized using attenuated total reflection Fourier transform infrared spectroscopy (ATR-FTIR), ellipsometry (for control samples of monocomponent unstructured brushes), and AFM. The molecular weight of PNIPAM and PtBA anchored to the surface was estimated using bulk polymers synthesized in the presence of the same initiator in the solutions. The molecular weight of PNIPAM (283 kg/mol) and PtBA (58.3 kg/mol) were measured using membrane osmometry and gel permeation chromatography (GPC), respectively. The grafting density for PNIPAM and PAA estimated using polymer brush thickness and molecular weight of the grafted polymers is 0.19±0.02 chains/nm² for both brushes.

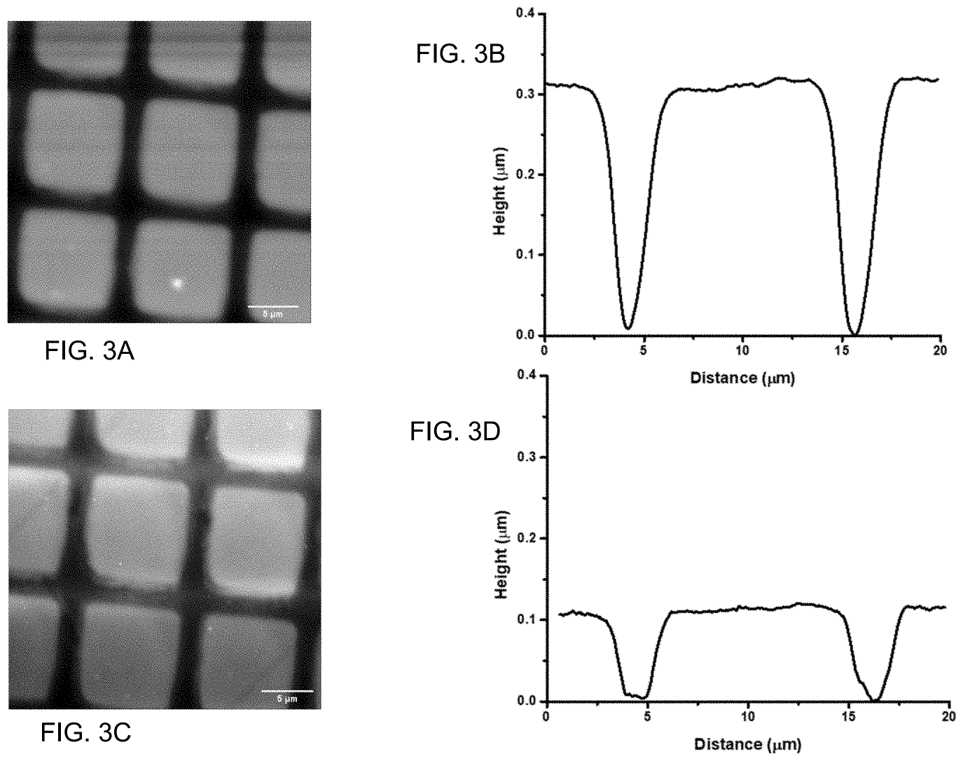

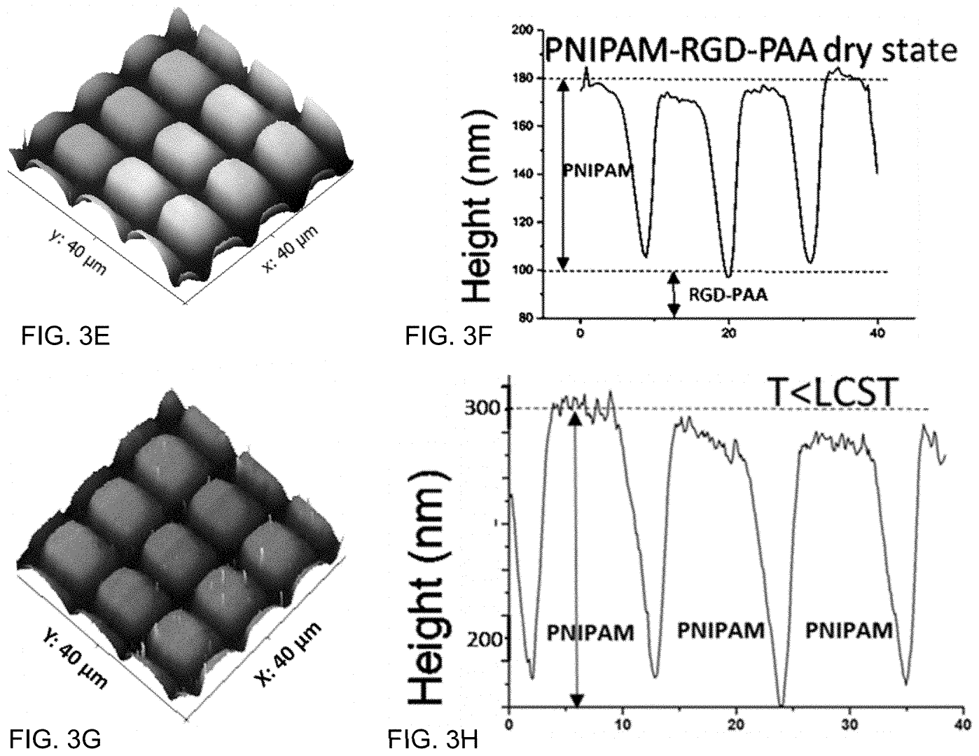

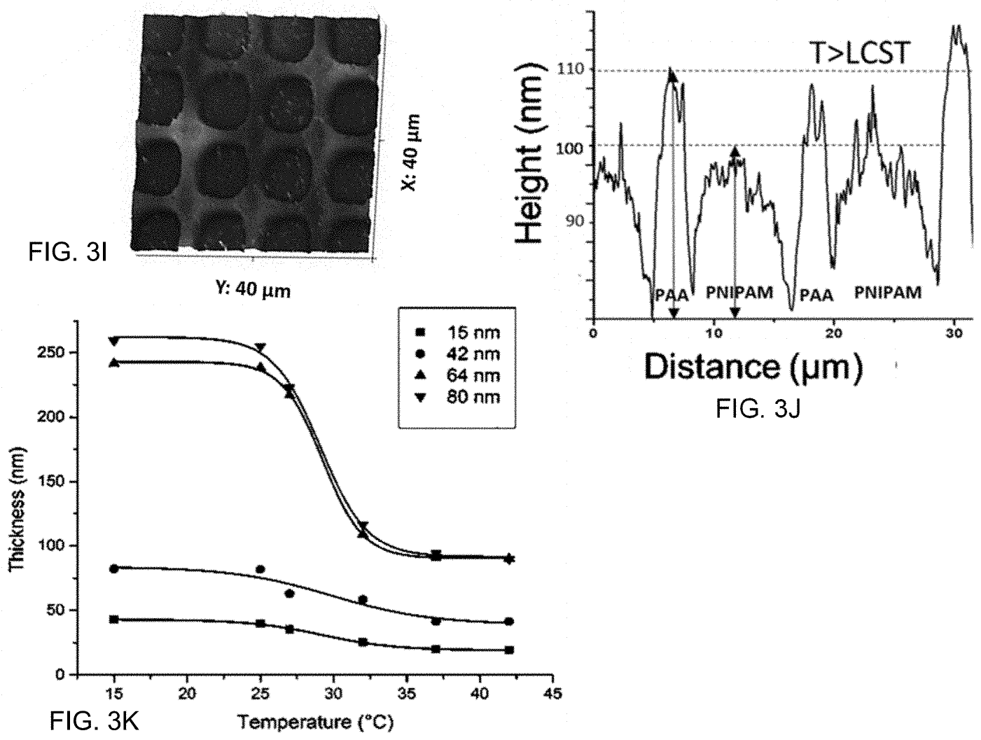

Stimuli-responsive behavior of the brush. For a representative example, FIGS. 3A-3J illustrate the PNIPAM brushes have a height of 100 nm in a PBS buffer at T>T_{LCST}, (FIG. 3C, 3D) while it expands to a 300 nm thickness at T<T_{LCST} (FIG. 3A, 3B). The height difference between the PNIPAM brush and RGD-PAA is 80 nm thick in the dry state (FIGS. 3 E, 3F). All thicknesses were evaluated with AFM and confirmed using ellipsometry and reference samples with one-component brushes. One-component PNIPAM microstructured brushes were used to quantify swelling of the PNIPAM domains by comparing the topographical profiles of the brush at a temperature below (FIG. 3A, 3B) and above (FIG. 3C, 3D) LCST. The temperature range for the transition between the swollen and collapsed state was verified using ellipsometry (FIG. 3K).

The RGD-PAA brush is 20 nm thick in the dry state and becomes swollen in a PBS buffer to a 110 nm thickness. At T>T_{LCST}, the RGD-PAA brush is 10 nm thicker than the PNIPAM brush (FIG. 31, 3J), while at T<T_{LCST}, the PNIPAM brush is 190 nm thicker than the RGD-PAA brush (FIG. 3G, 3H).

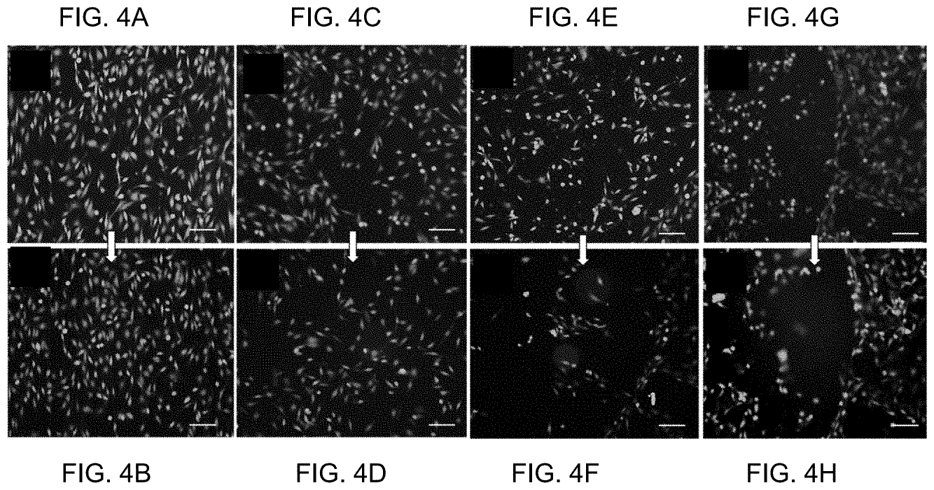

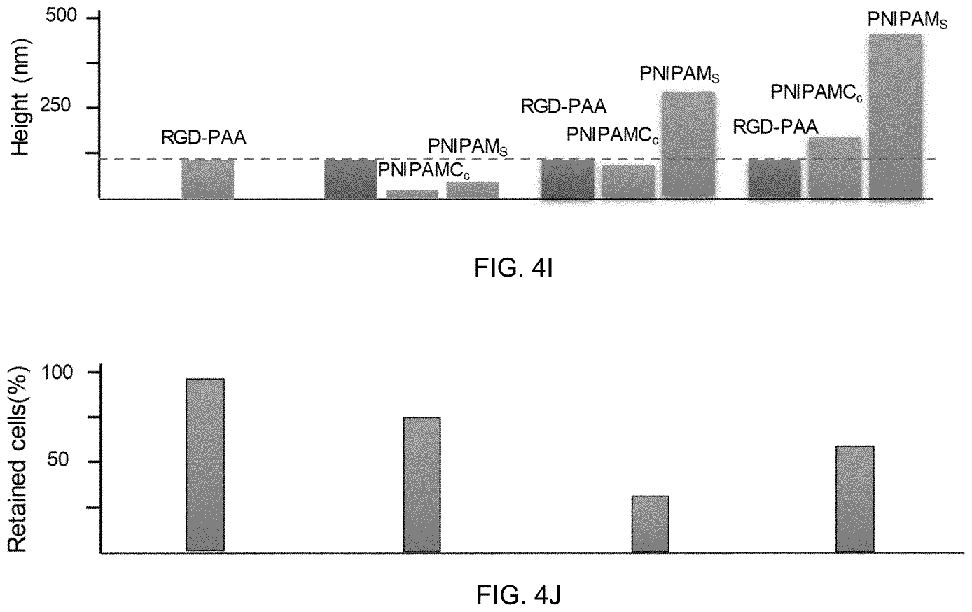

Swelling-collapse driven cell binding and detachment. NIH/3T3 fibroblast cells (15 μm average diameter) were used in the experiments to prove the potential of the microstructured polymer brush for temperature-triggered detachment of the particles (FIGS. 4A-4J). The effect of temperature on switching between adhesive and repulsive properties of the microstructured binary brush was evaluated by comparing it with a one-component RGD-PAA brush (FIGS. 4A, 4B, 4I, 4J) and with the microstructured brush when the PNIPAM domains are thicker than RGD-PAA domains even in the collapsed state (FIGS. 4 G, 4H, 4I, 4J). The cells captured by the one-component RGD-PAA brush remained on the surface even in conditions of periodic alternations of temperature around LCST due to no changes in RGD-PAA brush with temperature (FIGS. 4A, 4B, 4I, 4J).

However, a drop in temperature below LCST results in detachment of the cells captured by the binary microstructured brush (FIG. 4E, 4F, 4I, 4J). We consider interactions of particles of a radius R with polymer brushes. The simplest approximation is to assume that the shape of particles is not changed after adsorption. The latter is not always correct, for example, for the adsorption of live cells. To address this problem, we analyze two typical particle shapes: a spherical shape (typical for many colloids and live cells within a short period after adsorption) and a disk-like shape (less common for colloids but common for adherent cells in 1 h after adsorption).

At T>T_{LCST}, the cells adhere to the RGD-PAA brush. For the spherical shape, the adhesive force F_{As} can be estimated using the Derjaguin approximation based on interaction energy per cell surface area ω_{A}. The obtained in experiments with cells on RGD-decorated surfaces ω_{A}=1.25 10⁻¹⁵ J/m² in 10 min after adsorption (when the spherical shape of the cell is not substantially changed yet).²⁵ For the disk-like shape of colloids and cells (when cells spread over the surface and establish focal adhesion), the adhesive force F_{Ad} can be estimated based on the assumption of 100% cell-RGD-PAA contact using a typical experimentally measured²⁶ cell-protein-decorated interface stress constant, f_{SC}=5.5 nN/m².

At T<T_{LCST}, the repulsive force F_{B} is generated due to the osmotic pressure built in the PNIPAM brush. The repulsive force pushes the cells and it results in their detachment. The repulsive force F_{B} can be assessed experimentally by measuring the elastic modulus of the PNIPAM brush in repulsive domains, E=150 KPa.²⁷ For spherical particles, F_{B}=F_{Bs} was estimated using the Hertz theory. For disk-like particles, F_{B}=F_{Bd} was estimated using a model of the brush compressed between two undeformable surfaces. The simplified assessment considers no dependence of the elastic modulus on the compression, no deformation of colloids (cell membrane in the experiments with cells) upon interactions with the brush, zero strain of RGD-PAA brush upon swelling and pushing the particle by the PNIPAM brush, and no effect of the size and shape of the adhesive and repulsive domains as long as they are smaller than the particle diameter.

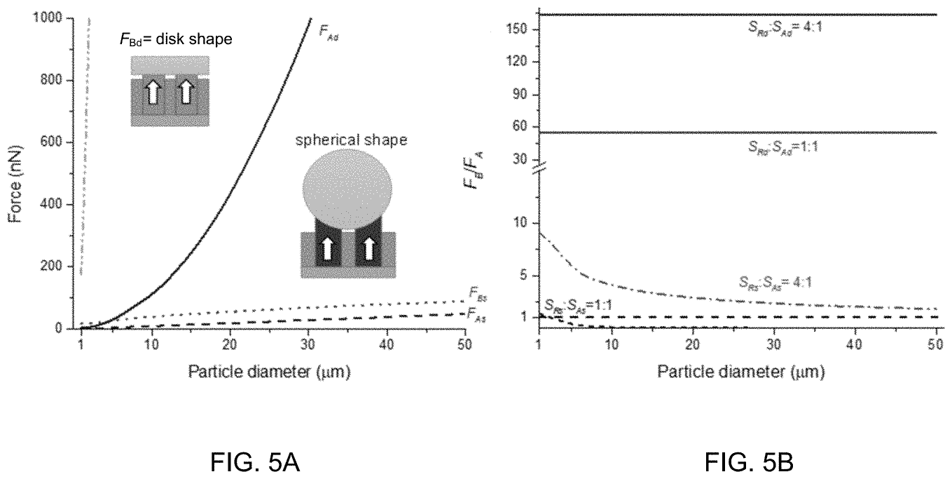

The results for colloids with a diameter greater than 1 μm and for the case of the brush with a fraction of adhesive domains φ_{A}=0.25 (corresponding to the microstructured polymer brush in our experiment) are shown in FIG. 5A. In the range of particle dimensions, the repulsive force exceeds the adhesion force for spherical and disk-like particles. Consequently, the particles are pushed and detached from the brush, as observed in the experiment with 3T3 cells. The range of F_{A} is in good agreement with experimentally reported data for F_{A}=1-1000 nN.²⁸ Obviously, disk-like shape particles adhere stronger at the interface than spherical particles due to the greater surface contact area.

The general conditions for the detachment can be expressed as the ratio of repulsive and adhesive forces F_{B}/F_{A}>1 (FIG. 4b):

$${⌊ \frac{{F}_{B}}{{F}_{A}} ⌋}_{s} = \frac{2 ( 1 - φ )}{3 φ ( 1 - {ϑ}^{2} )} \frac{{E}_{R} ( {δ}_{R} )}{{ω}_{A}} \sqrt{\frac{{δ}_{R}^{3}}{R}} ; {ɛ}_{A} = 0 ; for spherical particles$$

$${⌊ \frac{{F}_{B}}{{F}_{A}} ⌋}_{d} = \frac{( 1 - φ )}{φ ( 1 - {ϑ}^{2} )} \frac{{E}_{R} ( {ɛ}_{R} ) {ɛ}_{R}}{{f}_{S C}} ; {ɛ}_{A} = 0 ; for disk \text{-} like particles$$

where [symbol] is the Poisson ratio, ε_{R} is the compression strain of the repulsive domains, ε_{A} is the strain of adhesive domains, δ_{R} is the particle penetration depth in the repulsive brush domain, φ is the fraction of the surface area of the repulsive domains of the brush.

The efficiency of the detachment depends on φ (FIG. 5B) and polymer brush characteristics that can be used to adjust and regulate the F_{B}/F_{A} ratio. The adhesive force is adjusted by the selection of adhesive motifs and their concentration. The elastic modulus of PNIPAM brush is adjusted by varying the grafting density.²⁷ The penetration depth and the compression strain are adjusted by combinations of the height of the repulsive domains in the collapsed, H_{Bc}, and swollen state, H_{Bs} and the height of adhesive domains H_{A}. If H_{Bs}<H_{A}, the PNIPAM brush cannot reach the adhered cells at T<T_{LCST}. If H_{Bc}>H_{A}, the RGD-PAA brush cannot reach the cell at T>T_{LCST} (FIG. 4G, 4H).

For the latter case, we should note that many types of cells adhere to one-component PNIPAM thin films at T>T_{LCST}, but cells detach from the PNIPAM surface at T<T_{LCST} when PNIPAM becomes highly swollen in aqueous solutions.²⁹⁻³¹ However, in contrast to RGD motifs, this interaction is non-specific, and it is difficult to adjust the level of adhesion strength and target a specific type of cells or particles when cell adhesion/detachment is affected by non-specific interactions.³² Another important aspect is the kinetics of the detachment. As soon as T<T_{LCST} is reached, cells detach from the microstructured brush immediately, while the detachment from the PNIPAM surface for some cells is a very slow (20-40 min) process.³³⁻³⁵ A control experiment with 3T3 cells (FIG. 4G, 4H) demonstrates that the cells adhered to the PNIPAM brush only. However, a significant fraction of the adhered cells remained attached to the brush surface even after 1 h at T<T_{LCST}, likely due to long contact with the PNIPAM brush during the incubation period at T>T_{LCST}. This behavior contrasts with the rapid cell detachment (FIG. 4E, 4F) for the case when the cells were not in contact with PNIPAM domains during the incubation period.

Label-Free Refining Cells with the Stimuli-Responsive Brush.

We used the ability of dynamic microstructured interfaces to detach adsorbed particles for sorting and separation of colloids. In this example, we investigated the isolation of the rare circulating tumor cells (CTCs) from blood samples. As cancer progresses, cancer cells may escape from a primary solid tumor and enter the vascular system as CTCs, leading to dissemination of tumor cells throughout the body.³⁶ The detection of CTCs is often correlated with prognosis and survival of cancer patients.³⁷ The biological information carried by CTCs can provide important knowledge to understand the biology of metastasis and tumor progression that is needed to develop novel, more effective treatments.³⁸ A number of CTCs in 1 mL of peripheral blood ranges from 1 to 100 cells, which is much smaller compared with several billion red blood cells (RBCs) and several million white blood cells (WBCs). Despite the challenge, significant time and effort have been invested in establishing rare CTC isolation devices.³⁹⁻⁴¹ The current standard method is the tumor-antigen-dependent capture using epithelial cell adhesion molecules (EpCAM), such as the antibody-decorated magnetic particles of the CellSearch® system.⁴² EpCAM is overexpressed in several cancer cells such as breast, colon, stomach, head and neck, and lung cancers.⁴³⁻⁴⁵ The CellSearch® system detected CTCs in 52.6% of metastatic patients,⁴⁶ which demonstrated the need for improvements and developments of alternative methods. One of the reasons for false-negative results is masking of the surface of antibody-decorated magnetic particles because of non-specific adsorption of healthy blood cells.

The surface of CTC membrane is overexpressed with integrin that mediates interactions of cells with ECM. These proteins play a significant role in cancer cell progression and metastasis.^{47, 48} The integrin-RGD affinity has been broadly explored, including examples with RGD-modified polymer brushes,⁴⁹⁻⁵² for improving interactions with cells. Integrins are also expressed on the surface of healthy cells. However, at the very low ratio of CTCs to healthy cells, even if the polymer interface is decorated with integrin-specific motifs, it is indeed become populated predominantly by non-cancer cells. The adsorbed healthy cells block adsorption of CTCs that results in a decreased efficiency of the CTC isolation. A number of attempts to minimize interactions of healthy cells with polymer interfaces, for instance, by incorporating poly(ethylene oxide) (PEO) either through physisorption or chemisorption, have been reported, but these approaches also had limitations.⁵³⁻⁵⁶

The isolation of a small number of CTCs from billions of healthy cells circulating in the blood for diagnostic, research, and treatment purposes is an example of the problem of affinity-based separation of colloids that could be solved by multiple detachment-reattachment of blood cells until the CTCs are adsorbed due to their higher affinity to RGD-functionalized adsorbent. This specific task requires engineering of the brush surface when blood cells can be pushed and desorbed by the swollen PNIPAM domains while CTCs remain adsorbed. To achieve this, CTC adhesion force should be generally greater than the PNIPAM repulsive force, while blood cell adhesion force should be generally lower than the PNIPAM repulsive force.

We spiked human blood samples with a human glioblastoma cancer cell line, U-87MG, known for its high expression of integrins.⁵⁷ U-87MG cells were fixed with formalin after expansion and stained for fluorescence imaging. Healthy human blood cells were mixed with U-87MG cells at different ratios, ranging from 1:1000 to 1:10⁹.

First, we ran a series of reference experiments with stationary blood dispersions to exclude potential effects of drag forces on the experiments. The stationary media experiments were conducted in vials with introduced brush-decorated Si-wafers immersed in cell dispersions. The experiments with only U-87MG (data not shown) cells and only blood cells (data not shown) demonstrated that about 40% U-87MG and 100% of blood cells were detached. The results indicated a substantial difference in the surface affinity of cancer and mouse blood cells to the polymer brush sample.

For a 1:1000 mixture of cancer and blood cells, we repeated temperature cycles in the stationary conditions 9 times with one hour period to minimize convective flows. The fluorescent microscopy images were used to estimate the fraction of cancer cells collected on the brush surface as a function of a number of temperature cycles (data not shown). The cancer cell recovery reached >90% after 9 cycles. This experiment demonstrated that the sorting of the cells is caused by the stimuli-responsive brush with no effect or a minimal effect of liquid flow.

We used the liquid flow cell (FIG. 2D) with the microstructured polymer brush in a regime of periodic temperature oscillations above and below T_{LCST}. The geometry of liquid flow cell was designed to minimize the effect of a drag force on the detachment of the adsorbed cell but provide transport of cells in solutions for improved mass exchange between the interface and bulk. In the liquid flow cell, the cells are transported with an average speed of 5 cm/min. At this flow velocity, the detached and poorly adhered cells are rapidly transported away³³ and liberate adsorption sites on the surface. However, the drag force is lower than the adhesion force in this case; hence the desorption is caused mainly by the PNIPAM repulsive force.

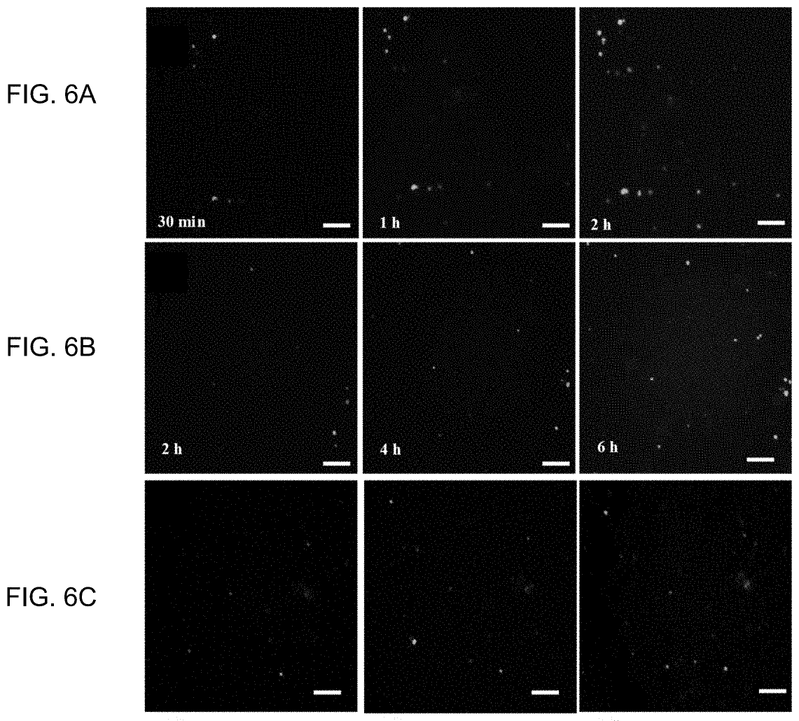

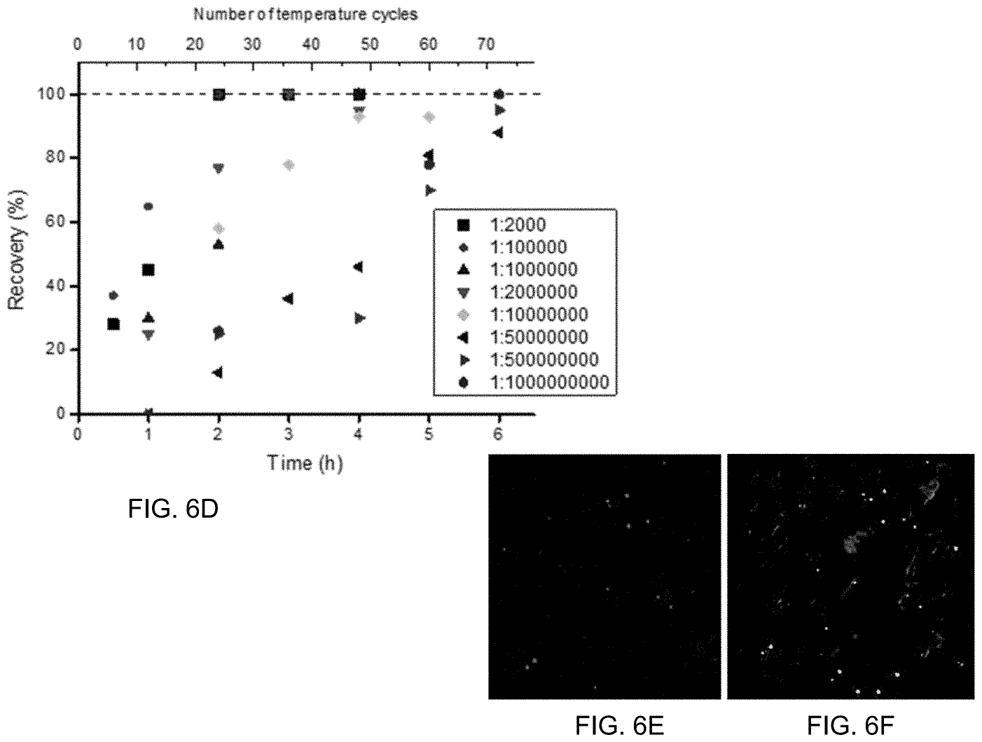

The efficiency of the CTCs separation as a function of a number of temperature cycles is shown in FIGS. 6A-6D. For all ratios of cell mixtures, 90 to 100% CTCs were successfully isolated and adhered to the microstructured polymer brush surface. There was a clear tendency for an increase in the number of temperature cycles with an increase in the dilution ratio. For a dilution ratio of 1 to 10⁹, 100% isolation was achieved after 72 temperature cycles in the 6 h experiment.

The analysis of the images (FIGS. 6A-6C) reveals that once CTCs were adsorbed on the brush surface, their positions were not changed after multiple temperature cycles. The result confirms the specific case of the separation when CTCs cells are strongly adsorbed on the surface and they cannot be detached by swollen PNIPAM domains. In contrast, the blood cells undergo multiple adsorption-desorption steps that facilitate CTCs to access the surface of the adsorbent.

The viability of the rectified cells was tested in a cell expansion experiment. Preserving functions is an important aspect of the separation. In the case of live cells, separation and isolation should not compromise their biological functions. To evaluate the feasibility of the developed method to preserve the biological functions of the sorted cells, we repeated the separation with healthy blood samples spiked with a live B16F10 murine melanoma cancer cells, which are transfected to express red fluorescence protein (RFP-B16F10), at a ratio of 1 to 5×10⁸. After 6 h refining, the sample of the micropatterned brush with the isolated cells was extracted from the fluid cell and transferred to a 24-well plate. After being cultured for 6 days, the sample was imaged under a fluorescence microscope (FIG. 6E, 6F). It was found that the cells more than double their number. The result demonstrates that the isolated cells retain their capacity for expansion.

### Conclusions/Discussion

In summary, the present example demonstrates development of a stimuli-responsive microstructured polymer brush interface, which is made of patterns of PNIPAM and RGD-PAA, for the separation and isolation of colloidal particles, including live and dead cells, based on their affinity to the brush surface. The developed interface showed highly effective sorting of target cells from a complex mixture with non-target cells even for highly asymmetric mixtures. The sorted cells retained their functions, which are often prone to damages that affect their viability and surface receptors by commonly used approaches. The present example provides new approaches for refining of colloids by their size, shape, and surface composition at different scales from laboratory to industrial production scale. This new approach provides advantages such as label-free isolation of cells, self-proliferation, and a facile detachment of cells without trypsinization.

### REFERENCES

1. Deblois, R. W.; Bean, C. P., Counting and sizing of submicron particles by resistive pulse technique. Rev. Sci. Instrum. 1970, 41 (7), 909-916.
2. Fulwyler, M. J., Electronic separation of biological cells by volume. Science 1965, 150 (3698), 910-911.
3. Plouffe, B. D.; Murthy, S. K.; Lewis, L. H., Fundamentals and application of magnetic particles in cell isolation and enrichment: a review. Rep. Prog. Phys. 2015, 78 (1), 1-38.
4. Fraikin, J. L.; Teesalu, T.; McKenney, C. M.; Ruoslahti, E.; Cleland, A. N., A high-throughput label-free nanoparticle analyser. Nat. Nanotechnol. 2011, 6 (5), 308-313.
5. Xia, N.; Hunt, T. P.; Mayers, B. T.; Alsberg, E.; Whitesides, G. M.; Westervelt, R. M.; Ingber, D. E., Combined microfluidic-micromagnetic separation of living cells in continuous flow.

Biomed. Microdevices 2006, 8 (4), 299-308.
6. Franke, T.; Braunmuller, S.; Schmid, L.; Wixforth, A.; Weitz, D. A., Surface acoustic wave actuated cell sorting (SAWACS). Lab Chip 2010, 10 (6), 789-794.
7. Pethig, R., Review article-dielectrophoresis: Status of the theory, technology, and applications. Biomicrofluidics 2010, 4 (2), 1-35.
8. Housmans, C.; Sferrazza, M.; Napolitano, S., Kinetics of irreversible chain adsorption. Macromolecules 2014, 47 (10), 3390-3393.
9. O'Shaughnessy, B.; Vavylonis, D., Irreversibility and polymer adsorption. Phys. Rev. Lett. 2003, 90 (5), 056103-1-056103-4.
10. Yu, C.; Granick, S., Revisiting Polymer Surface Diffusion in the Extreme Case of Strong Adsorption. Langmuir 2014, 30 (48), 14538-14544.
11. Aveyard, R.; Binks, B. P.; Clint, J. H., Emulsions stabilised solely by colloidal particles. Adv. Colloid Interface Sci. 2003, 100, 503-546.
12. Nagrath, S.; Sequist, L. V.; Maheswaran, S.; Bell, D. W.; Irimia, D.; Ulkus, L.; Smith, M. R.; Kwak, E. L.; Digumarthy, S.; Muzikansky, A.; Ryan, P.; Balis, U. J.; Tompkins, R. G.; Haber, D. A.; Toner, M., Isolation of rare circulating tumour cells in cancer patients by microchip technology. Nature 2007, 450 (7173), 1235-1239.
13. Poulichet, V.; Garbin, V., Ultrafast desorption of colloidal particles from fluid interfaces. Proc. Natl. Acad. Sci. USA 2015, 112 (19), 5932-5937.
14. Kurashina, Y.; Imashiro, C.; Hirano, M.; Kuribara, T.; Totani, K.; Ohnuma, K.; Friend, J.; Takemura, K., Enzyme-free release of adhered cells from standard culture dishes using intermittent ultrasonic traveling waves. Commun. Biol. 2019, 2 (393), 1-11.
15. Tan, I.; Roohi, F.; Titirici, M. M., Thermoresponsive polymers in liquid chromatography. Analytical Methods 2012, 4 (1), 34-43.
16. Kim, H.; Witt, H.; Oswald, T. A.; Tarantola, M., Adhesion of Epithelial Cells to PNIPAm Treated Surfaces for Temperature-Controlled Cell-Sheet Harvesting. Acs Applied Materials & Interfaces 2020, 12 (30), 33516-33529.
17. Garcia, A. J., Get a grip: integrins in cell-biomaterial interactions. Biomaterials 2005, 26 (36), 7525-7529.
18. Uhlig, K.; Boerner, H.; Wischerhoff, E.; Lutz, J.-F.; Jaeger, M.; Laschewsky, A.; Duschl, C., On the interaction of adherent cells with thermoresponsive polymer coatings. Polymers 2014, 6 (4), 1164-1177.
19. Plow, E. F.; Haas, T. A.; Zhang, L.; Loftus, J.; Smith, J. W., Ligand binding to integrins. J. Biol. Chem 2000, 275 (29), 21785-21788.
20. Humphries, J. D.; Byron, A.; Humphries, M. J., Integrin ligands at a glance. J. Cell Biol. 2006, 119 (19), 3901-3903.
21. Sandrin, L.; Thakar, D.; Goyer, C.; Labbe, P.; Boturyn, D.; Coche-Guerente, L., Controlled surface density of RGD ligands for cell adhesion: evidence for ligand specificity by using QCM-D. J. Mater. Chem. B 2015, 3 (27), 5577-5587.
22. Ionov, L.; Minko, S., Mixed polymer brushes with locking switching. ACS Appl. Mater. Interfaces 2012, 4 (1), 483-489.
23. Poloukhtine, A. A.; Mbua, N. E.; Wolfert, M. A.; Boons, G.-J.; Popik, V. V., Selective labeling of living cells by a photo-triggered click reaction. J. Am. Chem. Soc. 2009, 131 (43), 15769-15776.
24. Laradji, A. M.; McNitt, C. D.; Yadavalli, N. S.; Popik, V. V.; Minko, S., Robust, Solvent-Free, Catalyst-Free Click Chemistry for the Generation of Highly Stable Densely Grafted Poly(ethylene glycol) Polymer Brushes by the Grafting To Method and Their Properties. Macromolecules 2016, 49 (20), 7625-7631.
25. Sztilkovics, M.; Gerecsei, T.; Peter, B.; Saftics, A.; Kurunczi, S.; Szekacs, I.; Szabo, B.; Horvath, R., Single-cell adhesion force kinetics of cell populations from combined label-free optical biosensor and robotic fluidic force microscopy. Sci. Rep. 2020, 10 (1), 1-13.
26. Ladoux, B.; Nicolas, A., Physically based principles of cell adhesion mechanosensitivity in tissues. Rep. Prog. Phys. 2012, 75 (11), 1-25.
27. Sui, X. F.; Chen, Q.; Hempenius, M. A.; Vancso, G. J., Probing the Collapse Dynamics of Poly(N-isopropylacrylamide) Brushes by AFM: Effects of Co-nonsolvency and Grafting Densities. Small 2011, 7 (10), 1440-1447.
28. Sagvolden, G.; Giaever, I.; Pettersen, E. O.; Feder, J., Cell adhesion force microscopy. Proc. Natl. Acad. Sci. U.S.A. 1999, 96 (2), 471-476.
29. Patel, N. G.; Cavicchia, J. P.; Zhang, G.; Newby, B. M. Z., Rapid cell sheet detachment using spin-coated pNIPAAm films retained on surfaces by an aminopropyltriethoxysilane network. Acta Biomater. 2012, 8 (7), 2559-2567.
30. Yang, L.; Fan, X. G.; Zhang, J.; Ju, J., Preparation and Characterization of Thermoresponsive Poly(N-lsopropylacrylamide) for Cell Culture Applications. Polymers 2020, 12 (2), 1-24.
31. Nagase, K.; Yamato, M.; Kanazawa, H.; Okano, T., Poly(N-isopropylacrylamide)-based thermoresponsive surfaces provide new types of biomedical applications. Biomaterials 2018, 153, 27-48.
32. Doberenz, F.; Zeng, K.; Willems, C.; Zhang, K.; Groth, T., Thermoresponsive polymers and their biomedical application in tissue engineering—a review. J. Mater. Chem. B 2020, 8 (4), 607-628.
33. Ernst, O.; Lieske, A.; Jager, M.; Lankenau, A.; Duschl, C., Control of cell detachment in a microfluidic device using a thermo-responsive copolymer on a gold substrate. Lab Chip 2007, 7 (10), 1322-1329.
34. Tang, Z.; Akiyama, Y.; Itoga, K.; Kobayashi, J.; Yamato, M.; Okano, T., Shear stress-dependent cell detachment from temperature-responsive cell culture surfaces in a microfluidic device. Biomaterials 2012, 33 (30), 7405-7411.
35. Afif, S.; Ghaleh, H.; Nasiri, M.; Maher, B. M.; Abbasi, F., Adhesion, proliferation, and detachment of cells on poly (N-isopropyl acrylamide) brushes tethered on polystyrene using surface-initiated atom transfer radical polymerization. Mater. Today Commun. 2020, 25, 1-7.
36. Aceto, N.; Toner, M.; Maheswaran, S.; Haber, D. A., En Route to Metastasis: Circulating Tumor Cell Clusters and Epithelial-to-Mesenchymal Transition. Trends Cancer 2015, 1 (1), 44-52.
37. Park, Y.; Jun, H. R.; Choi, H. W.; Hwang, D. W.; Lee, J. H.; Song, K. B.; Lee, W.; Kwon, J.; Ha, S. H.; Jun, E., Circulating tumour cells as an indicator of early and systemic recurrence after surgical resection in pancreatic ductal adenocarcinoma. Sci. Rep. 2021, 11 (1), 1-12.
38. Yu, M.; Stott, S.; Toner, M.; Maheswaran, S.; Haber, D. A. J. T. J. o. c. b., Circulating tumor cells: approaches to isolation and characterization. J. Cell Biol. 2011, 192 (3), 373-382.
39. Arya, S. K.; Lim, B.; Rahman, A. R. A., Enrichment, detection and clinical significance of circulating tumor cells. Lab Chip 2013, 13 (11), 1995-2027.
40. Yue, W.-Q.; Tan, Z.; Li, X.-P.; Liu, F.-F.; Wang, C., Micro/nanofluidic technologies for efficient isolation and detection of circulating tumor cells. TRAC-Trend Anal Chem. 2019, 117, 101-115.
41. Opoku-Damoah, Y.; Assanhou, A. G.; Sooro, M. A.; Baduweh, C. A.; Sun, C.; Ding, Y., Functional diagnostic and therapeutic nanoconstructs for efficient probing of circulating tumor cells. ACS Appl. Mater. Interfaces 2018, 10 (17), 14231-14247.
42. Litvinov, S. V.; Velders, M. P.; Bakker, H.; Fleuren, G. J.; Warnaar, S. O., Ep-CAM: a human epithelial antigen is a homophilic cell-cell adhesion molecule. J. Cell Biol. 1994, 125 (2), 437-446.
43. Yu, M.; Bardia, A.; Wittner, B. S.; Stott, S. L.; Smas, M. E.; Ting, D. T.; Isakoff, S. J.; Ciciliano, J. C.; Wells, M. N.; Shah, A. M., Circulating breast tumor cells exhibit dynamic changes in epithelial and mesenchymal composition. Science 2013, 339 (6119), 580-584.
44. Nichols, A. C.; Lowes, L. E.; Szeto, C. C.; Basmaji, J.; Dhaliwal, S.; Chapeskie, C.; Todorovic, B.; Read, N.; Venkatesan, V.; Hammond, A., Detection of circulating tumor cells in advanced head and neck cancer using the CellSearch system. Head neck 2012, 34 (10), 1440-1444.
45. Went, P.; Vasei, M.; Bubendorf, L.; Terracciano, L.; Tornillo, L.; Riede, U.; Kononen, J.; Simon, R.; Sauter, G.; Baeuerle, P., Frequent high-level expression of the immunotherapeutic target Ep-CAM in colon, stomach, prostate and lung cancers. Br. J. Cancer 2006, 94 (1), 128-135.
46. Politaki, E.; Agelaki, S.; Apostolaki, S.; Hatzidaki, D.; Strati, A.; Koinis, F.; Perraki, M.; Saloustrou, G.; Stoupis, G.; Kallergi, G.; Spiliotaki, M.; Skaltsi, T.; Lianidou, E.; Georgoulias, V.; Mavroudis, D., A Comparison of Three Methods for the Detection of Circulating Tumor Cells in Patients with Early and Metastatic Breast Cancer. Cellular Physiology and Biochemistry 2017, 44 (2), 594-606.
47. Ruoslahti, E., RGD and other recognition sequences for integrins. Annu. Rev. Cell Dev. Biol. 1996, 12 (1), 697-715.
48. Nieberler, M.; Reuning, U.; Reichart, F.; Notni, J.; Wester, H.-J.; Schwaiger, M.; Weinmuller, M.; Rader, A.; Steiger, K.; Kessler, H., Exploring the role of RGD-recognizing integrins in cancer. Cancers 2017, 9 (116), 1-33.
49. Tugulu, S.; Silacci, P.; Stergiopulos, N.; Klok, H.-A., RGD—Functionalized polymer brushes as substrates for the integrin specific adhesion of human umbilical vein endothelial cells. Biomaterials 2007, 28 (16), 2536-2546.
50. Groll, J.; Fiedler, J.; Engelhard, E.; Ameringer, T.; Tugulu, S.; Klok, H. A.; Brenner, R. E.; Moeller, M., A novel star PEG-derived surface coating for specific cell adhesion. J. Biomed. Mater. Res. A 2005, 74 (4), 607-617.
51. Petrie, T. A.; Raynor, J. E.; Reyes, C. D.; Burns, K. L.; Collard, D. M.; Garcia, A. J., The effect of integrin-specific bioactive coatings on tissue healing and implant osseointegration. Biomaterials 2008, 29 (19), 2849-2857.
52. Muszanska, A. K.; Rochford, E. T.; Gruszka, A.; Bastian, A. A.; Busscher, H. J.; Norde, W.; van der Mei, H. C.; Herrmann, A., Antiadhesive polymer brush coating functionalized with antimicrobial and RGD peptides to reduce biofilm formation and enhance tissue integration. Biomacromolecules 2014, 15 (6), 2019-2026.
53. Park, K. D.; Kim, W. G.; Jacobs, H.; Okano, T.; Kim, S. W., Blood compatibility of SUUU-PEO-heparin graft copolymers. J. Biomed. Mater. Res. 1992, 26 (6), 739-756.
54. Amiji, M.; Park, K., Surface modification of polymeric biomaterials with poly (ethylene oxide), albumin, and heparin for reduced thrombogenicity. J. Biomater. Sci. Polym. Ed. 1993, 4 (3), 217-234.
55. Harris, J. M., Introduction to biotechnical and biomedical applications of poly (ethylene glycol). In Poly (ethylene glycol) Chemistry, Springer: 1992; pp 1-14.
56. Irvine, D.; Mayes, A.; Satija, S.; Barker, J.; Sofia-Allgor, S.; Griffith, L., Comparison of tethered star and linear poly (ethylene oxide) for control of biomaterials surface properties. J. Biomed. Mater. Res. 1998, 40 (3), 498-509.
57. Nakada, M.; Nambu, E.; Furuyama, N.; Yoshida, Y.; Takino, T.; Hayashi, Y.; Sato, H.; Sai, Y.; Tsuji, T.; Miyamoto, K., Integrin α3 is overexpressed in glioma stem-like cells and promotes invasion. Br. J. Cancer 2013, 108 (12), 2516-2524.

### Example 2—Microstructured Thermoresponsive Polymeric Interface for Cell Proliferation and Spontaneous Harvest

### Introduction

Cell culture is considered as an essential tool for studying cellular and molecular biology¹, various regenerative medicine and tissue engineering applications², production of biopharmaceuticals³⁻⁶, stem cell research^{7, 8}, and many other biotechnological researches. Two cell culture techniques are broadly used to grow cells in culture: monolayers on an artificial flat surface, which is termed as monolayer culture or adherent culture; and cells in a free-floating culture medium, which is called suspension culture.⁹ Most of the tissues used for biotechnological research are composed of adherent cells. Although some cell lines are adaptive to both techniques, the suspension cell culture method requires higher maintenance and more frequent monitoring, thus making it difficult to maintain a consistent culture quality. Therefore, the adherent cell culture technique has been used as is the predominant culture technique. This technique requires detachment and collection before redeposition and adhesion, called cell passage.¹⁰ To detach the cells from the culture dish or flask, peptide bonds in proteins need to be cut, which can be done by proteolytic enzymes, such as trypsin, acuutate, and dispase. However, as discussed above, the use of such a harvesting method can damage extracellular matrix (ECM) components containing cell-binding proteins and cell membrane receptors, which are directly related to cell activity potential and functionality of treated tissue.^{11, 12} The cell viability can be affected by prolonged treatment of trypsin, initiating cytotoxicity.^{13, 14}

Therefore, detachment and collection of cells from culture substances without hampering the functionality of cells and disturbing the microenvironment has always been a pressing issue in clinical and research applications; not only for standard cell culture but also for culturing rare and whole-cell and for molecular profiling.^{15, 16} Many techniques have been proposed as an alternative of trypsinization, including shear stress, using pH-responsive culture surface^{17, 18} introducing ion-induced cell sheet¹⁹, the light-triggered release of cells^{20, 21}, electrochemical desorption^{22, 23}, surface acoustic wave²⁴, Surface modification²⁵, acoustic pressure²⁶⁻²³ temperature-responsive polymers.²⁹⁻³¹ Innovative cell detachment methods have accelerated the development of biotechnological fields, such as biopharmaceuticals, tissue engineering, gene modification, etc. As there are many different methods available from various backgrounds, a competitive method is required.

Thermoresponsive polymers, such as poly(N-isopropyl acrylamide) (PNIPAM), have been used to in cell engineering as a thermoresponsive surface by changing a physical property from hydrophobic state to hydrophilic state at the low critical solution temperature (LCST) of 32° C.^{31, 32} PNIPAM brushes can have hydrophilic properties below T_{LCST} and hydrophobic property above T_{LCST}. Cells prefer more hydrophobic surfaces than hydrophilic surfaces. For this reason, cells can adhere and spread on the PNIPAM surface at 37° C., but they leave the surface at a temperature below T_{LCST}. Cell detachment from thermoresponsive surface avoids the type of damages caused by conventional enzyme utilized cell detachment method.

Unfortunately, not all PNIPAM based surfaces are favorable for the applications of cell adhesion, proliferation, and spontaneous detachment. Thus, cell adhesion on the PNIPAM surface is not observed even above T_{LCST}, making it undesirable as a cell scaffold substrate.^{33, 34} The thickness or grafting density of PNIPAM brushes are referred to as common limitation factors that determine the success of PNIPAM based cell detachment. It appears that a thickness thicker than 35-40 nm (grafting density >2.9 μg/cm²) inhibits cell adherence on the surface as a physical phase transition of PNIPAM at the temperature below T_{LCST} results in incomplete dehydration of PNIPAM brushes, which results in a less hydrophobic surface.³⁵ Also, depending on cell types, not all cells can be used on such surfaces. Lastly, as competitive and convenient surfaces are needed, an all-in-one surface, having multifunctional capabilities for cell adhesion, isolation, proliferation, and detachment are desired.

Macrophages are heterogeneous innate immune cells recruited for the detection and primary defense of the host response to pathogens. Macrophages can be found migrating and circulating within every organ and tissue, playing roles in coordinating the adaptive immune response, tissue homeostasis, and inflammatory response.^{36, 37} Macrophages can detect invaders through a system of recognition receptors capable of directly or indirectly signaling to modify the cells' functional properties, inducing specialized activation programs like gene and protein expression patterns.³⁸

Macrophages can radically change their form and physiology in response to environmental stimuli, referred to as the “activation” response of macrophages.³⁹ Most of the studies on macrophages have been carried out in the context of host defense. Therefore, the “activation” responses have been strongly correlated with the immune effector functions of the host.⁴⁰ Cell-mediated immunity research has demonstrated the significance of activated macrophages as crucial immune effector cells.⁴¹ The macrophage activation profile can be affected by pathway-specific transcription factors and receptors, cytosolic enzymes, functionally distant genes, and many other elements. Identification of the factors and mechanisms affecting the activation profile of macrophages are important for designing cell-specific therapeutics, drugs, and many immunological researches.⁴²

Endotoxin is a lipopolysaccharide (LPS) containing lipid and a polysaccharide made of O-antigen. LPS is the conserved component of the exterior membrane of gram-negative bacteria like E. coli and is considered one of the most effective microbial initiators of inflammation.⁴³ LPS can initiate the signaling pathways of macrophages, enabling the macrophages to release immunoregulatory molecules that recruit and activate other immune cells to assist in fighting the invaders. Also, other wide-ranging biological response mediators, such as platelet-activating factors, enzymes, and free radicals like nitric oxide,^{44, 45} can be released by LPS induced macrophages. Also, the LPS-induced macrophages exhibit increased expression of adhesion molecules.⁴⁶

However, trypsin can inhibit LPS signaling by obstructing the LPS-induced nitric oxide production. Trypsin can repress various protein expressions, degrade multiple proteins, and accessory molecules of proteins.⁴⁷ Therefore, the conventional method of detaching the LPS-induced macrophages from the test specimen, which uses trypsin, can affect research results. Hence, an enzyme-free cell detachment method is vital to study the LPS-induced macrophages without any signaling inhibitor element.

This present example describes development of an embodiment of an enzyme-free cell detachment surface of the present disclosure having two distinctive adhesive and repulsive domains of microstructure materials. The developed interface provides an alternative surface between adhesive and repulsive domains. When the interface is of an adhesive characteristic, cells approach the interface and adhere to adhesive domains. However, cells are spontaneously detached by the precise tuning of the repulsive domain and temperature oscillation. The approach of this concept utilizes a thermoresponsive polymer, PNIPAM, to generate a repulsive force so that cells adhering to the adhesive domain and proliferating along the interface can be pushed away from the surface by reaching the equilibrium state between cell adhesion and desorption. This is demonstrated using RAW 264.7 cells, a monocyte/macrophage cell line.

### Results

Design of Stimuli-Responsive Interfaces with Oscillating Repulsive Domains for Spontaneous Cell Detachment System.

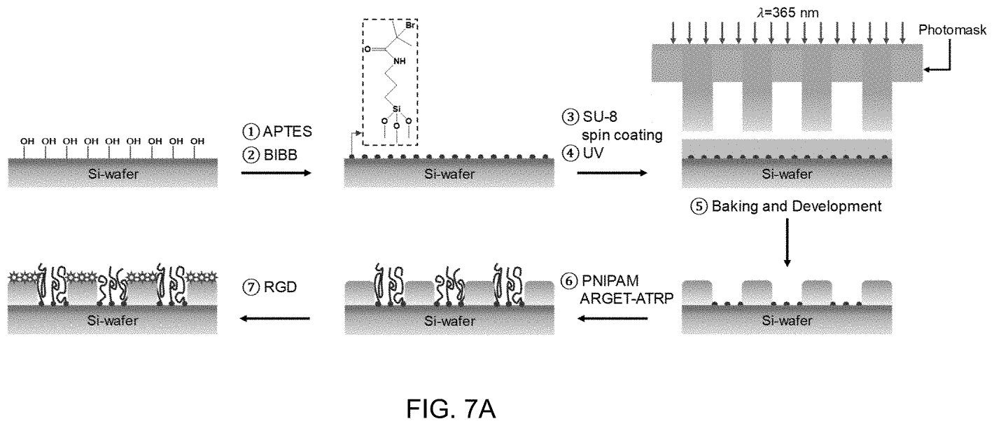

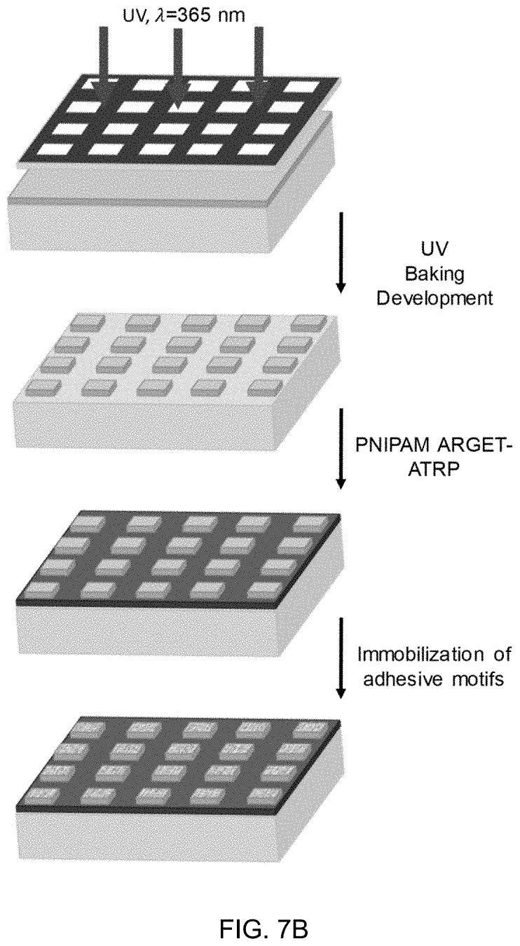

The microstructured polymer interface, shown in FIGS. 7A-7B, is composed of two different domains representing grid pattern geometry. The process of making the microstructured polymer interface is illustrated in FIG. 7A, with FIG. 7B illustrating a different view of the use of photoresist to fabricate the patterned surface. First, repulsive domains are made of PNIPAM grafted from the surface-initiated Si-wafer substrates. Adhesive domains indicate the square shape of the island on the surface, fabricated using SU-8 photoresist as explained elsewhere. Then, the surface of adhesive domains is conjugated with cell-adhesive peptides, Arg-Gly-Asp (RGD). The RGD is known for a commonly used cell-adhesive peptide sequence that is presented in extracellular matrix (ECM) proteins that bind to integrins on the cell membrane.⁴⁸⁻⁵⁰ The strength of cell adhesion to various types of interfaces can be controlled by the concentration of RGD on the surface. The PNIPAM is known for experiencing a phase transition at LCST, T_{LCST}=32° C. The polymer brush collapses and becomes hydrophobic above T_{LCST}, whereas the brush is hydrated and stretches itself from a surface of the substrate below T_{LCST} in an aqueous media. Based on this temperature-responsive mechanism, PNIPAM domains in these embodiments provide the cell-repulsive domains and generate repulsive forces to push away adsorbed cells by changing their volume.

PNIPAM domains and RGD conjugated adhesive domains are considered as cell-repulsive domains and cell-adhesive domains in this example, respectively. To realize this mechanism and effectively control the cell adsorption and desorption, height differences between two domains are designed. The PNIPAM has a substantially higher thickness than the thickness of adhesive domain at a temperature below T_{LCST}. Thus, cells on the interface are repelled from the interface and are not able to approach adhesive domains at this temperature. However, when the temperature is above T_{LCST}, the height of PNIPAM is lower than the thickness of adhesive domain. At this temperature, cells are able to approach the interface and bind to RGD conjugated adhesive domain. This temperature oscillation process allows cells to adhere on the interface and let them proliferate at the temperature above T_{LCST}, and spontaneously detaches cells after proliferation without enzyme treatment at room temperature, which is the temperature below T_{LCST}.

### Synthesis of Polymeric Microstructured PNIPAM-RGD@SU-8 Interface

Grafting Polymer brushes and patterning coating were carried out using an activator regenerated by electron transfer atom transfer radical polymerization (ARGET-ATRP) and SU-8 photoresist, respectively, on Si-wafer substrates as described in Ionov, L. et al., 2012⁵¹, which is hereby incorporated by reference herein. As illustrated in FIG. 7A, the surface of Si-wafers was functionalized with aminosilane groups using APTES, followed by immobilizing α-bromosiobutyryl bromide (BIBB), ATRP initiator, to graft NIPAM monomers. Prior to the polymerization of PNIPAM, an adhesive domain was fabricated using SU-8 2002 photoresist under the lithographed photomask with 4 μm square holes and 4 μm bar width, as illustrated in FIG. 7B. The SU-8 2002 photoresist provides epoxy functional groups, which enable the conjugation of proteins. The thickness of the adhesive domain is 80 (±5 nm) nm measured by atomic force microscopy (AFM). After the fabrication of adhesive domains, PNIPAM brushes were grafted only on non-adhesive domain areas because initiators under the adhesive domains were blocked by photoresists, so NIPAM monomers could not access to initiators under adhesive domains. The thickness of PNIPAM brushes was 58 (±5) nm and 55 (±0.4) nm measured by AFM and ellipsometry, respectively. Finally, RGD cell-adhesive motifs were conjugated on adhesive domains directly through epoxy-amine ring-opening reaction. The thickness of adhesive domains increased up to 3 nm (±0.3) measured by ellipsometry.

### Stimuli-Responsive Behavior of Interface

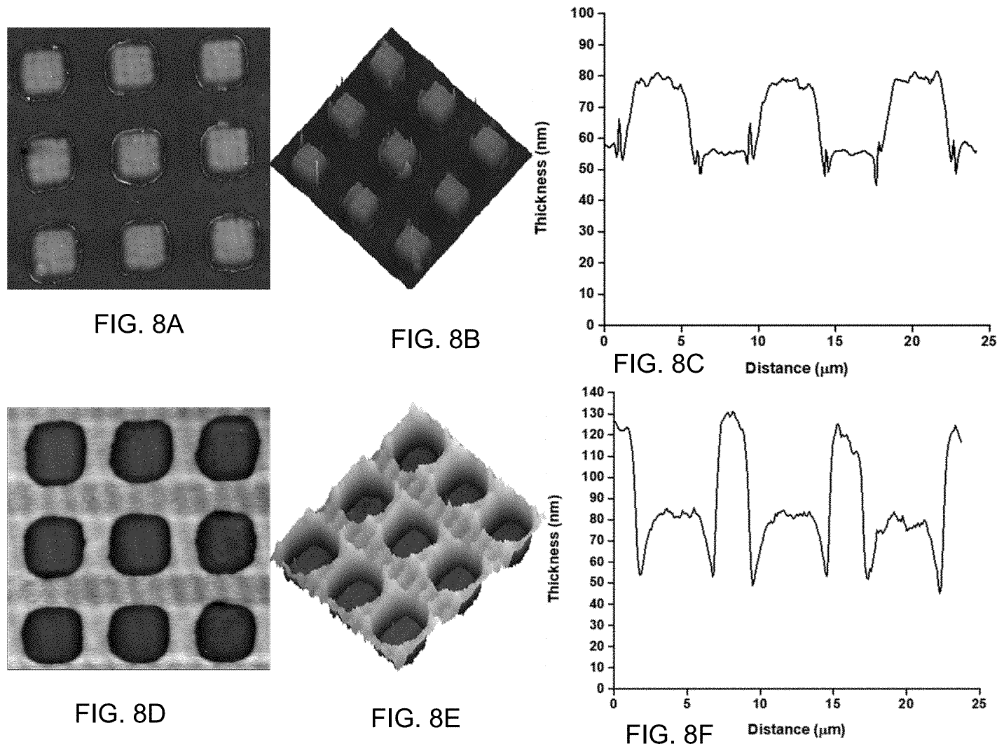

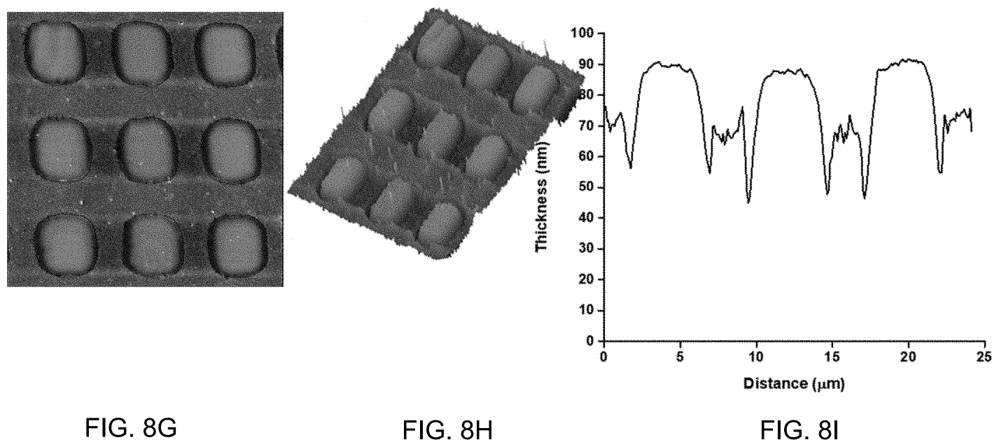

The actual topography of the proposed interface is shown in the images of FIGS. 8A, 8B, 8D, 8E, 8G, and 8H. As illustrated in the graphs of FIGS. 8C, 8F, and 8I, the achieved thickness of the PNIPAM brush is 58 nm thick in a dry state (FIG. 8C), 68 nm in a PBS buffer at T>T_{LCST} (FIG. 8I), while it expands to a 130 nm thickness at T<T_{LCST} (FIG. 8F). The height differences between the adhesive domain and the PNIPAM domain were monitored under the aqueous media using AFM. The RGD@SU-8 domains are 80 nm thick in both dry and aqueous states. At T>T_{LCST}, the RGD@SU-8 domain is approximately 20 nm thicker than the PNIPAM brush, whereas the PNIPAM brush is approximately 50 nm thicker than the RGD@SU-8 domain at T<T_{LCST}.

The effect of temperature on switching between adhesive and repulsive interface provides a new strategy of cell adhesion and detachment. Cells can approach the surface and adhere to the adhesive domains when the PNIPAM brushes are collapsed at T>T_{LCST} when the height of the adhesive domain is higher than the repulsive domain, allowing the cells to interact with the RGD adhesive motif on the adhesive domains. However, a decrease in temperature below T_{LCST} results in the swelling of the PNIPAM brushes and detachment of cells from the surface when the PNIPAM brushes stretch and push cells from the surface as the brushes are hydrated and hydrophilic. The detached cells also do not adhere to PNIPAM brushes. In embodiments, the repulsive forces generated from PNIPAM brushes can overcome the force between adhered cells and adhesive domain, thereby detaching/releasing cells are detached from the microstructured interface.

At T>T_{LCST}, the cells adhere to the RGD-PAA brush adhesive domains. In embodiments, cells, or other particles can have various shapes, and soft colloid particles (including cells) can change shape in response to forces, such as discussed above in Example 1 and illustrated in FIGS. 5A and 5B.

It is noted that many types of cells adhere to one-component PNIPAM thin films at T>T_{LCST}, but cells detach from the PNIPAM surface at T<T_{LCST} when PNIPAM becomes highly swollen in aqueous solutions.⁵⁶⁻⁵⁸ However, in contrast to surfaces with the adhesive domains with RGD motifs, this interaction is non-specific, and it is difficult to adjust the level of adhesion strength and target a specific type of cells or particles when cell adhesion/detachment is affected by non-specific interactions.⁵⁹ Another important aspect is the kinetics of the detachment. As soon as T<T_{LCST} is reached, cells detach from the microstructured dynamic polymer brush immediately, while the detachment from PNIPAM-only surface for some cells is a very slow process.⁶⁰⁻⁶² An experiment with 3T3 cells (see Example 1) demonstrated that the cells adhere to the PNIPAM brush and the cells remained attached to the brush surface even after 1 h at T<T_{LCST}.

### Cell Detachment

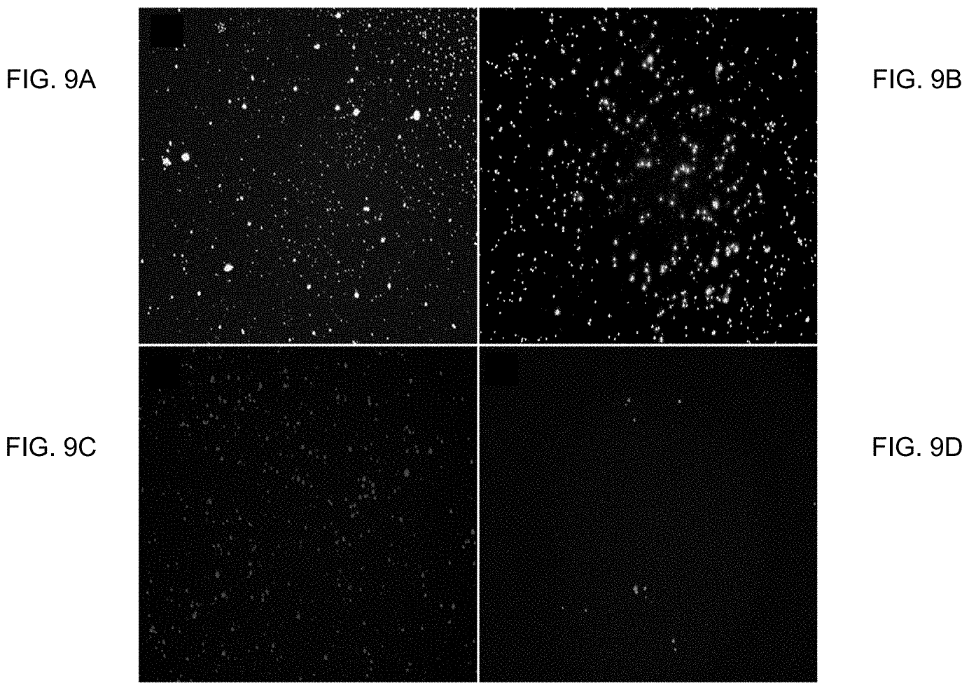

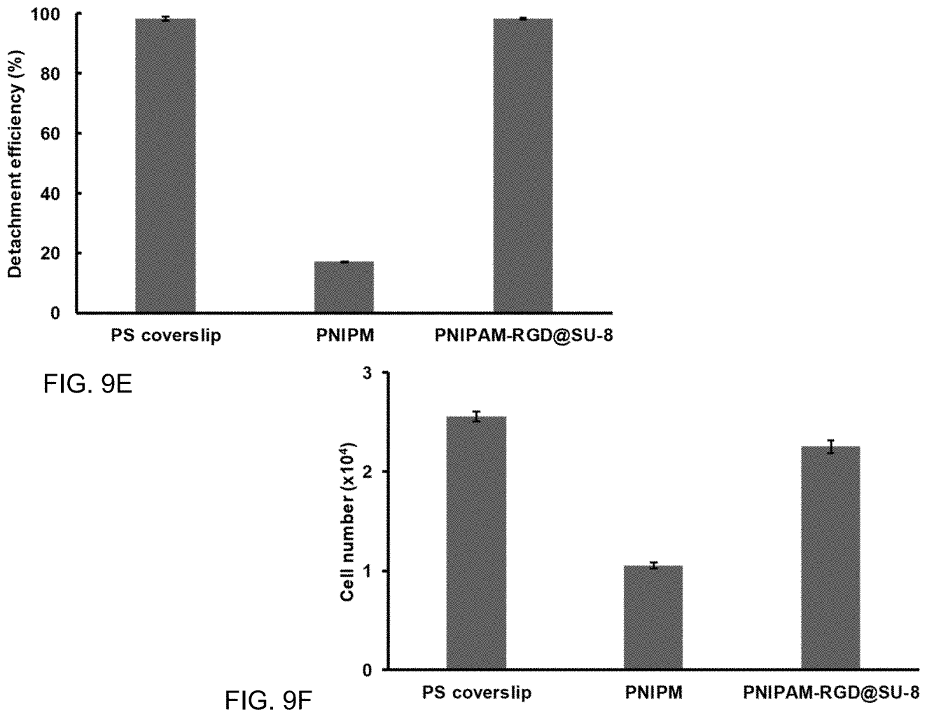

The cell detachment capability of the proposed method was compared with the conventional trypsinization method as illustrated in FIGS. 9A-9F. LPS activated RAW 264.7 cells, a monocyte/macrophage cell line, were seeded onto the 12-well plates containing polystyrene (PS) coverslip, the PNIPAM grafted substrate, and PNIPAM-RGD@SU-8 substrate, then cultured in a medium for 48 h. LPS was added into each well before starting incubation and the concentration of LPS was 0.1 μg/L. After incubation, detached cells by trypsinization and the proposed method were evaluated. First, the number of cells was counted by the fluorescent signal using fluorescent microscopy and is illustrated in FIGS. 9A and 9B. Remaining cells on substrates were monitored again after cell detachment (FIGS. 9C and 9D). As illustrated in FIG. 9F, the new proliferation/detachment method with the dynamic interface showed that no significant difference in the number of proliferated cells compared to PS coverslip, which is treated for optimized cell culture. However, it showed significant improvement compared to the PNIPAM monolayer, which showed about 60% less number of cells even though the thickness of PNIPAM was optimized thickness for cell proliferation. FIG. 9E illustrates that cell detachment efficiency of PNIPAM-RGD@SU-8 showed 98.4% which is comparable to that with trypsin treatment, 98.5%, but significantly higher than PNIPAM monolayer interface. Its efficiency was 16.9%, which was 6 times less than the proposed method and trypsinization method. The new detachment method proves that its ability to proliferate and detach cells are comparable enough to the results of the conventional method. Lastly, cells that are not favorable to the PNIPAM surface can grow better and be detached spontaneously by temperature oscillation.

### MTT and Alamar Blue Viability Assay of Detached Cells

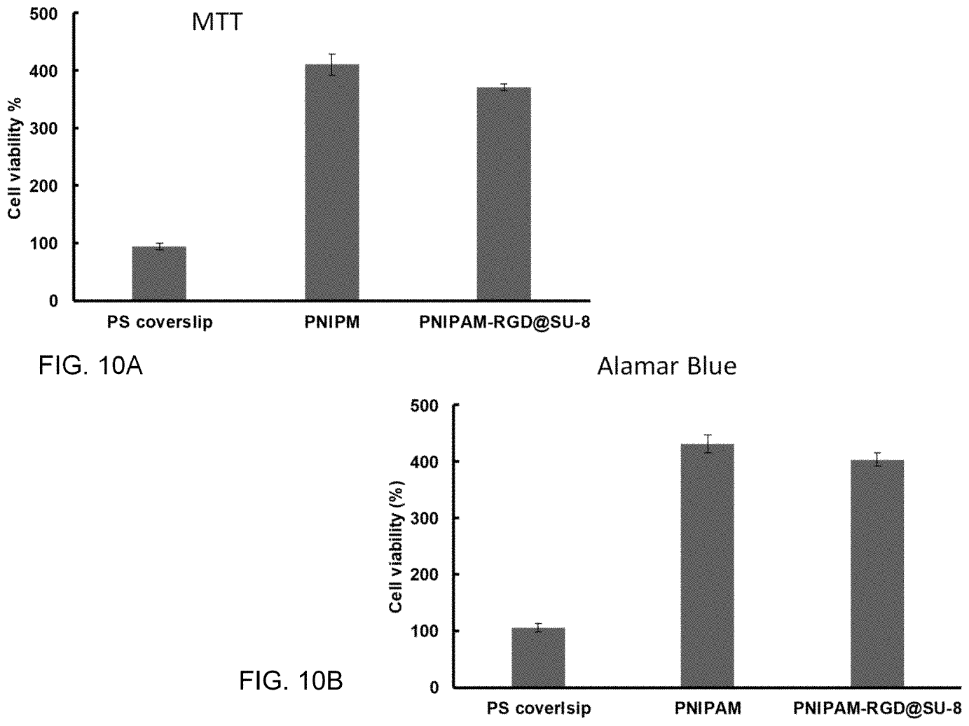

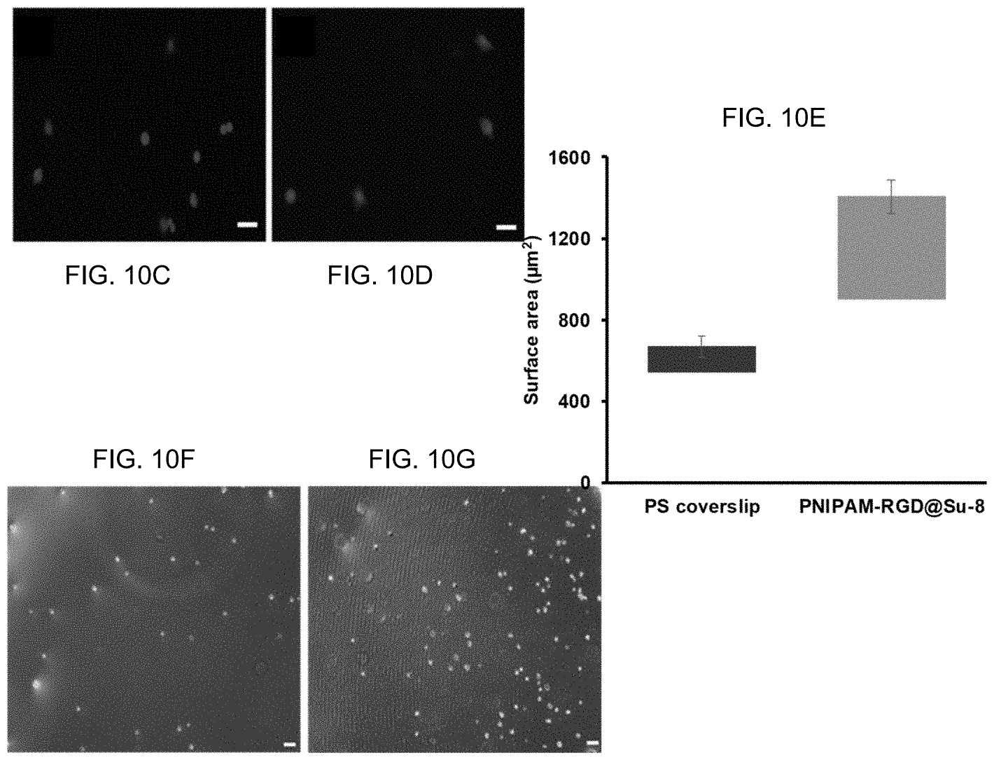

Metabolic activity of detached cells was evaluated by MTT and Alamar Blue assay. Alamar Blue assay and MTT assay were performed to compare the detached cell's viability and proliferation as illustrated in FIGS. 10A-10G. The results shown in FIGS. 10A and 10B illustrate the increment of detached cell viability and proliferation of cells detached via the trypsinization method from a PS coverslip vs detachment from PNIPM monolayer and the new physical detachment methods via activation of responsive repulsive domains. The MTT data shows that when the cells were detached from the PNIPAM monolayer substrate, there are 4.4 times viable cells compared to the trypsinized cells detached from the PS coverslip, while there are almost 4 times of higher viable cells in the PNIPAM-RGD@SU-8 method (FIG. 10A). The Alamar blue data also supports the viability results found by MTT. The viable cells in PNIPAM monolayer detached sample were 4 times higher than the cells detached from trypsinized method, while there are almost 3.82 times of increment of cell viability using the PNIPAM-RGD@SU-8 sample (FIG. 10B). This data indicates the cytotoxic effect of trypsinized method.

### Characterization of Detached Cells

Conserving crucial cell proteins, such as actins, for cellular function and motility is essential for detached cells to be used for further applications. F-actin is cytoskeletal actin that manages cell stability and morphogenesis, thus having undamaged F-actin is vital for the detached cells to maintain their functionality. The f-actin was evaluated by comparing the surface area of f-actin of cells detached by the conventional method (e.g. trypsin) and from the dynamic polymer surfaces of the present disclosure (FIGS. 10C, 10D, and 10E). The F-actin proteins of detached cells were stained, and images were taken using the IVIS 100 (IVIS Spectrum imaging system) (FIGS. 10C (trypsinization) and 10D (method of present disclosure).

The images show that the sizes and shapes of cells detached using the proposed method are more prominent and sturdier than cells detached with trypsinization, which indicates damage of cytoskeleton protein F-actin in the trypsinization method. The surface area of cells detached from the PNIPAM-RGD@Su-8 dynamic surface by the proposed method (1290.17 μm²) was twice that of the cells detached from the PS coverslip by the conventional method (603.14 μm²). This indicates that the new method of detachment using dynamic polymer surfaces can conserve cell proteins, preserving cell functionality wherein the trypsinization method, cells were damaged. The result was further proved through a reattachment test. The post-adhesion of detached cells to a new surface is observed by reseeding detached cells, as shown in FIGS. 10F and 10G as a comparison between the two detachment methods. The number of adhered cells after 10 min of incubation was 3 times greater for cells previously detached using the proposed method (FIG. 10G) than for cells detached using trypsinization (FIG. 10F). This is likely because damage to surface proteins of cells detached by the dynamic surface was less than for the cells detached by trypsinization.

### Materials

Si-wafers were purchased from University Wafer, Boston, Mass., USA. (3-aminopropyl) triethoxysilane (APTES), N-isopropylacrylamide (97%) (NIPAM), ascorbic acid (ASCO), α-bromosiobutyryl bromide (BIBB), triethylamine (TEA), copper (II) bromide (CuBr₂), N, N, N′, N″, N″-pentamethyldiethylenetriamine (PMDTA), RGD peptide, and cyclopentanone were purchased from Millipore Sigma. Sulfuric acid, dichloromethane, and 30% hydrogen peroxide (H₂O₂) were purchased from Fisher Scientific. NIPAM monomers were recrystallized in hexane to remove inhibitors prior to the polymerization. For fabrication of microstructured patterns, SU-8 2002 and SU-8 developer were purchased from Kayaku Advanced Materials, Inc. For cell experiments, RAW 264.7 cell line, Resazurin sodium salt (Alamar Blue), Hanks' Balanced Salt solution (HBSS), Phosphate buffered saline (PBS), Lipopolysaccharide (LPS from E. coli O8:K27), Calcein-AM, Ethidium Homodimer-1, Rhodamine Phalloidin, Hoechst 33342 were purchased from Sigma Aldrich (USA). MTT (3-(4,5-Dimethylthiazol-2-yl)-2, Dimethyl Sulfoxide (DMSO) were purchased from Thermo fisher Scientific. Dulbecco's modified eagle's media-high glucose (DMEM), antibiotics and antimycotics, fetal bovine serum (FBS), 0.25% Trypsin-EDTA, glutaraldehyde, Triton X-100 were obtained from VWR International LLC (USA). For cell culture and cell assay, standard T-75 treated flasks, cell culture treated 24-well plates, flat-bottom cell culture treated 96-well plates were used, which were purchased from VWR International LLC (USA). wheat germ agglutinin (WGA), and Vybrant™ DID were received from Thermofisher Scientific. Lithographed photomask was received from the Oak Ridge National lab.

### Methods

Measurements.

Atomic Force Microscopy (AFM) images were obtained using the Bruker Multimode Nanoscope MM8 in tapping mode. Brush thickness was estimated with a Nanofilm ep4 imaging null-ellipsometer, Accurion Inc. (Germany) and AFM. A microscope (Olympus BX51) was used to take images of microstructured patterns of the surface. Scepter™ 3.0 handheld cell counter were used to determine the number of cells. IVIS 100 (IVIS Spectrum imaging system) was used for imaging fluorescent signals from cells.

Functionalization of Surface and Fabrication of Microstructured Patterns

The 0.9×0.9 cm² of Si-wafer substrates were cleaned in piranha solution, 3:1 ratio of sulfuric acid and H₂O₂, for 30 min at 90° C. Cleaned Si-wafers were rinsed with DI water and ethanol, followed by drying under the flux of argon. Then, substrates were put in the 2% of APTES solution in ethanol for 24 h to functionalize the surface with amino groups. Initiators for the polymerization of NIPAM monomer were immobilized by incubating substrates in the 2% of TEA and 1% of BIBB in anhydrous dichloromethane for 3 h at the room temperature. Substrates were rinsed with ethanol and dried under the argon. Prior to the polymerization of NIPAM monomer, microstructured pattern domains, which are the support of adhesive domains, are fabricated on the substrate. The 11% of SU-8 2002 in cyclopentanone was spin-coated on the initiator immobilized substrate at 4000 rpm with 500 rpm/s for 40 sec. Introduced SU-8 film is soft-baked at 65° C. for 2 min and then, exposed to UV-light (365 nm, 7 mW/cm²) for 15 sec under the photomask. Post exposure bake of the sample was carried out after the exposure on the hotplate, 65° C. for 2 min, followed by the development of the sample in SU-8 developer for 30 sec. Finally, the sample was rinsed with 2-propanol and dried under the argon gas. The thickness of pattern was measured after a hard bake at 125° C. for 30 min.

Polymerization of NIPAMs and Conjugation of RGD on Adhesive Domains

The polymerization of NIPAMs was carried out by ARGET-ATRP on the microstructured pattern introduced sample. PNIPAMs grew from only non-adhesive areas, where initiators are available because initiators at adhesive domains are blocked by SU-8 photoresist. The sample was placed in a solution of 40 wt % of recrystallized NIPAMs in 300 ul of DI water and 700 ul of methanol in 20 ml of a vial. 9 μl of 0.22 M CuBr₂ and 9 μl of 0.48 M PMDTA were added into the solution and oxygen was removed by purging the solution with argon gas. Then, 50 μL of ASCO (0.04 g/ml) was added into polymerization reactors slowly; it was sealed. The polymerization was proceeded by placing the vial in 35 C of water bath for 30 min. The polymerization was stopped by opening the cap and the sample was rinsed with ethanol to remove residual of monomers and other chemicals.

RGD was conjugated to the adhesive domain through epoxy-amine reaction. The RGD solution is prepared by dissolving 0.125 mg in 1 ml of PBS buffer, pH 7.4. The 50 μL of RGD solution was loaded on sample and the conjugation was proceed for 6 h. Finally, samples were washed with PBS pH 7.4 and sterilized prior to use for a cell proliferation scaffold.

Cell Culture

RAW 264.7 Macrophages were cultured with Dulbecco's Modified Eagle's Medium (DMEM), supplemented with 200 U/mL penicillin and 200 mg/mL streptomycin, and 10% fetal bovine serum (FBS). The cells were incubated at 37° C. and 5% CO₂ and sub-cultured at about 80% confluency.

Cell Proliferation and Harvest

The RAW 264.7 cell, a monocyte/macrophage cell line, was used as a representative adherent cell line, The RAW 264.7 cells were cultured in different types of polymeric interfaces and PS coverslip as reference. PS coverslips along with various types of polymer interfaces, were placed in a sterile 24-well plate, and RAW 264.7 macrophages were seeded at a density around 2.5×10⁴ cells per well with 1 mL DMEM. Cell were incubated for 48 h in a 5% CO₂ humidified atmosphere incubator at 37° C. After desired incubation time, cells were used for proposed cell detachment method to compare with conventional trypsinization method. The samples were washed gently with warm (37° C.) PBS to remove the non-adherent cells. Cells on PS coverslip were collected through conventional trypsinization method, while cells on polymeric interfaces were transferred to a sterile 24-well plate containing 1 mL of fresh culture medium, followed by room temperature incubation for 10 min for temperature-stimuli cell detachment. Then, detached cells were collected in 1.5 mL tubes and used for analysis. The number of cells detached from proposed interfaces and PS coverslip were counted using Scepter™ 3.0 handheld cell count. The number of cells remaining on each substrate were counted by the immunostaining method. Each sample was stained by green fluorescent Calcein-AM and red-fluorescent ethidium homodimer-1 in PBS. Calcein-AM indicates intracellular esterase activity, which indicates the live cells, and Ethidium Homodimer-1 indicates the loss of plasma membrane integrity, showing dead cells. After 20 mins of incubation, the fluorescence images are taken using IVIS 100 (IVIS Spectrum imaging system). For each type of reference and proposed samples, triplicates were used.

Cell Viability Assay

Alamar blue and MTT assays were performed to compare detached cells' viability after detachment with conventional trypsinization and the proposed dynamic surface methods. The detached cells collected from each sample were treated with Alamar Blue solution (0.15 mg/mL in PBS, pH=7.4) at 10% volume of cell culture medium and incubated for 3 h at 37° C. The active ingredient of Alamar Blue is a non-fluorescent compound, Resazurin, which is reduced to resorufin, a red and highly fluorescent compound after entering the living cells that is directly proportional to the number of living cells. The fluorescence was measured at excitation at 540 nm and emission at 590 nm using Varioskan LUX Multimode Microplate Reader.

For the MTT assay, the collected cells from each sample were treated with [3-(4,5-dimethylthiazol-2-yl)-2,5-diphenyltetrazolium bromide] (MTT) solution (5 mg/mL in PBS) and incubated for 3 h at 37° C. After 3 h of incubation, culture supernatants were aspirated, and insoluble purple colored MTT product formazan crystal was dissolved in dimethyl sulfoxide (DMSO) for 15 minutes. The amount of produced formazan is directly proportional to the number of metabolically active viable cells. After that, absorbance was measured at the wavelength of 570 nm using Varioskan LUX Multimode Microplate Reader. For both assays, 3 repeats were done for each kind of sample.

Cell Adhesion Area Assay and Initial Adhesion Assay

Initially, detached cells from the proposed dynamic interface using temperature oscillation and from the PS coverslip by trypsinization were reseeded in a sterile 96-well plate with culture medium and incubated for 10 min. Then, the old medium was replaced by fresh culture medium and the adhered cells were counted to evaluate the initial adhesion of cells using IVIS 100 (IVIS Spectrum imaging system).

F-Actin Assay

The detached cells collected by conventional trypsinization method and proposed method were reseeded with fresh culture medium to a sterile 96-well plate and allowed to be attached for 1 h. Then the cells were fixed with 2.5% glutaraldehyde for 15 mins in room temperature. 0.1% Triton X-100 was used to permeabilize the cells. Then the cells were stained with rhodamine phalloidin in PBS (50 μg/mL) for 30 mins to stain F-actin filaments and incubated in room temperature in dark. After washing with PBS, cell nuclei were stained using Hoechst 33342 (0.1 μg/ml) for 10 minutes. Then, the fluorescence images were taken using IVIS 100 (IVIS Spectrum imaging system) and the area of F-actin was analyzed using ImageJ.

### REFERENCES FOR EXAMPLE 2

1. Pampaloni, F.; Reynaud, E. G.; Stelzer, E. H. K., The third dimension bridges the gap between cell culture and live tissue. Nature Reviews Molecular Cell Biology 2007, 8 (10), 839-845.
2. Mao, A. S.; Mooney, D. J., Regenerative medicine: Current therapies and future directions. Proceedings of the National Academy of Sciences 2015, 112 (47), 14452.
3. Dumont, J.; Euwart, D.; Mei, B.; Estes, S.; Kshirsagar, R., Human cell lines for biopharmaceutical manufacturing: history, status, and future perspectives. Crit Rev Biotechnol 2016, 36 (6), 1110-1122.
4. Kim, J. Y.; Kim, Y.-G.; Lee, G. M., CHO cells in biotechnology for production of recombinant proteins: current state and further potential. Applied Microbiology and Biotechnology 2012, 93 (3), 917-930.
5. Lazarovici, P.; Li, M.; Perets, A.; Mondrinos, M. J.; Lecht, S.; Koharski, C. D.; Bidez lii, P. R.; Finck, C. M.; Lelkes, P. I., Intelligent Biomatrices and Engineered Tissue Constructs: In-Vitro Models for Drug Discovery and Toxicity Testing. In Drug Testing in vitro, 2006; pp 1-51.
6. Zhu, J., Mammalian cell protein expression for biopharmaceutical production. Biotechnology Advances 2012, 30 (5), 1158-1170.
7. Baker, M., Stem cells in culture: defining the substrate. Nature Methods 2011, 8 (4), 293-297.
8. McKee, C.; Chaudhry, G. R., Advances and challenges in stem cell culture. Colloids and Surfaces B: Biointerfaces 2017, 159, 62-77.
9. Harrison, M. A.; Rae, I. F., General Techniques of Cell Culture. Cambridge University Press: 1997.
10. Freshney, R. I., Culture of animal cells: a manual of basic technique and specialized applications. John Wiley & Sons: 2015.
11. Hayman, D. M.; Blumberg, T. J.; Scott, C. C.; Athanasiou, K. A., The effects of isolation on chondrocyte gene expression. Tissue engineering 2006, 12 (9), 2573-2581.
12. Huang, H.-L.; Hsing, H.-W.; Lai, T.-C.; Chen, Y.-W.; Lee, T.-R.; Chan, H.-T.; Lyu, P.-C.; Wu, C.-L.; Lu, Y.-C.; Lin, S.-T.; Lin, C.-W.; Lai, C.-H.; Chang, H.-T.; Chou, H.-C.; Chan, H.-L., Trypsin-induced proteome alteration during cell subculture in mammalian cells. J Biomed Sci 2010, 17 (1), 36-36.
13. Brown, M. A.; Wallace, C. S.; Anamelechi, C. C.; Clermont, E.; Reichert, W. M.; Truskey, G. A., The use of mild trypsinization conditions in the detachment of endothelial cells to promote subsequent endothelialization on synthetic surfaces. Biomaterials 2007, 28 (27), 3928-3935.
14. Foglieni, C.; Meoni, C.; Davalli, A. M., Fluorescent dyes for cell viability: an application on prefixed conditions. Histochemistry and cell biology 2001, 115 (3), 223-229.
15. den Toonder, J., Circulating tumor cells: the Grand Challenge. Lab on a Chip 2011, 11 (3), 375-377.
16. Zheng, Q.; Iqbal, S. M.; Wan, Y., Cell detachment: Post-isolation challenges. Biotechnology Advances 2013, 31 (8), 1664-1675.
17. Chen, Y.-H.; Chung, Y.-C.; Wang, I. J.; Young, T.-H., Control of cell attachment on pH-responsive chitosan surface by precise adjustment of medium pH. Biomaterials 2012, 33 (5), 1336-1342.
18. Guillaume-Gentil, O.; Semenov, O. V.; Zisch, A. H.; Zimmermann, R.; Vörös, J.; Ehrbar, M., pH-controlled recovery of placenta-derived mesenchymal stem cell sheets. Biomaterials 2011, 32 (19), 4376-4384.
19. Zahn, R.; Thomasson, E.; Guillaume-Gentil, O.; Vörös, J.; Zambelli, T., Ion-induced cell sheet detachment from standard cell culture surfaces coated with polyelectrolytes. Biomaterials 2012, 33 (12), 3421-3427.
20. Hong, Y.; Yu, M.; Weng, W.; Cheng, K.; Wang, H.; Lin, J., Light-induced cell detachment for cell sheet technology. Biomaterials 2013, 34 (1), 11-18.
21. Pasparakis, G.; Manouras, T.; Selimis, A.; Vamvakaki, M.; Argitis, P., Laser-induced cell detachment and patterning with photodegradable polymer substrates. Angewandte Chemie 2011, 123 (18), 4228-4231.
22. Inaba, R.; Khademhosseini, A.; Suzuki, H.; Fukuda, J., Electrochemical desorption of self-assembled monolayers for engineering cellular tissues. Biomaterials 2009, 30 (21), 3573-3579.
23. Zhu, H.; Yan, J.; Revzin, A., Catch and release cell sorting: Electrochemical desorption of T-cells from antibody-modified microelectrodes. Colloids and Surfaces B: Biointerfaces 2008, 64 (2), 260-268.
24. Bok, M.; Li, H.; Yeo, L. Y.; Friend, J. R., The dynamics of surface acoustic wave-driven scaffold cell seeding. Biotechnology and bioengineering 2009, 103 (2), 387-401.
25. Albrecht, D. R.; Underhill, G. H.; Wassermann, T. B.; Sah, R. L.; Bhatia, S. N., Probing the role of multicellular organization in three-dimensional microenvironments. Nature methods 2006, 3 (5), 369-375.
26. Haake, A.; Neild, A.; Radziwill, G.; Dual, J., Positioning, displacement, and localization of cells using ultrasonic forces. Biotechnology and bioengineering 2005, 92 (1), 8-14.
27. Lee, J.; Lee, C.; Kim, H. H.; Jakob, A.; Lemor, R.; Teh, S. Y.; Lee, A.; Shung, K. K., Targeted cell immobilization by ultrasound microbeam. Biotechnology and bioengineering 2011, 108 (7), 1643-1650.
28. Kurashina, Y.; Imashiro, C.; Hirano, M.; Kuribara, T.; Totani, K.; Ohnuma, K.; Friend, J.; Takemura, K., Enzyme-free release of adhered cells from standard culture dishes using intermittent ultrasonic traveling waves. Communications biology 2019, 2 (1), 1-11.
29. Ernst, O.; Lieske, A.; Jager, M.; Lankenau, A.; Duschl, C., Control of cell detachment in a microfluidic device using a thermo-responsive copolymer on a gold substrate. Lab on a Chip 2007, 7 (10), 1322-1329.
30. Liu, H.; Liu, X.; Meng, J.; Zhang, P.; Yang, G.; Su, B.; Sun, K.; Chen, L.; Han, D.; Wang, S., Hydrophobic interaction-mediated capture and release of cancer cells on thermoresponsive nanostructured surfaces. Advanced Materials 2013, 25 (6), 922-927.
31. Mokhtarinia, K.; Masaeli, E., Transiently thermally responsive surfaces: Concepts for cell sheet engineering. European Polymer Journal 2020, 110076.
32. Ward, M. A.; Georgiou, T. K., Thermoresponsive polymers for biomedical applications. Polymers 2011, 3 (3), 1215-1242.
33. Akiyama, Y.; Kikuchi, A.; Yamato, M.; Okano, T., Ultrathin poly (N-isopropylacrylamide) grafted layer on polystyrene surfaces for cell adhesion/detachment control. Langmuir 2004, 20 (13), 5506-5511.
34. Yamato, M.; Konno, C.; Koike, S.; Isoi, Y.; Shimizu, T.; Kikuchi, A.; Makino, K.; Okano, T., Nanofabrication for micropatterned cell arrays by combining electron beam-irradiated polymer grafting and localized laser ablation. Journal of Biomedical Materials Research Part A: An Official Journal of The Society for Biomaterials, The Japanese Society for Biomaterials, and The Australian Society for Biomaterials and the Korean Society for Biomaterials 2003, 67 (4), 1065-1071.
35. Da Silva, R. M.; Mano, J. F.; Reis, R. L., Smart thermoresponsive coatings and surfaces for tissue engineering: switching cell-material boundaries. TRENDS in Biotechnology 2007, 25 (12), 577-583.
36. Gordon, S., Alternative activation of macrophages. Nature Reviews Immunology 2003, 3 (1), 23-35.
37. Martinez, F. O.; Helming, L.; Gordon, S., Alternative activation of macrophages: an immunologic functional perspective. Annual review of immunology 2009, 27, 451-483.
38. Martinez, F. O.; Gordon, S.; Locati, M.; Mantovani, A., Transcriptional profiling of the human monocyte-to-macrophage differentiation and polarization: new molecules and patterns of gene expression. The Journal of Immunology 2006, 177 (10), 7303-7311.
39. Cohn, Z. A., The activation of mononuclear phagocytes: fact, fancy, and future. The journal of immunology 1978, 121 (3), 813-816.
40. Celada, A.; Nathan, C., Macrophage activation revisited. Immunology today 1994, 15 (3), 100-102.
41. Mosser, D. M.; Zhang, X., Activation of murine macrophages. Curr Protoc Immunol 2008, Chapter 14, Unit-14.2.
42. Martinez, F. O., Regulators of macrophage activation. European Journal of Immunology 2011, 41 (6), 1531-1534.
43. Cohen, J., The immunopathogenesis of sepsis. Nature 2002, 420 (6917), 885-891.
44. Cheng, Y.-W.; Cheah, K.-P.; Lin, C.-W.; Li, J.-S.; Yu, W.-Y.; Chang, M. L.; Yeh, G.-C.; Chen, S.-H.; Choy, C.-S.; Hu, C.-M., Myrrh mediates haem oxygenase-1 expression to suppress the lipopolysaccharide-induced inflammatory response in RAW264. 7 macrophages. Journal of Pharmacy and Pharmacology 2011, 63 (9), 1211-1218.
45. Hambleton, J.; Weinstein, S. L.; Lem, L.; DeFranco, A. L., Activation of c-Jun N-terminal kinase in bacterial lipopolysaccharide-stimulated macrophages. Proceedings of the National Academy of Sciences 1996, 93 (7), 2774-2778.
46. Leporatti, S.; Gerth, A.; Köhler, G.; Kohlstrunk, B.; Hauschildt, S.; Donath, E., Elasticity and adhesion of resting and lipopolysaccharide-stimulated macrophages. FEBS Letters 2006, 580 (2), 450-454.
47. Komatsu, H.; Shimose, A.; Shimizu, T.; Mukai, Y.; Kobayashi, J.; Ohama, T.; Sato, K., Trypsin inhibits lipopolysaccharide signaling in macrophages via toll-like receptor 4 accessory molecules. Life Sciences 2012, 91 (3), 143-150.
48. Garcia, A. J., Get a grip: integrins in cell-biomaterial interactions. Biomaterials 2005, 26 (36), 7525-7529.
49. Humphries, J. D.; Byron, A.; Humphries, M. J., Integrin ligands at a glance. Journal of cell science 2006, 119 (19), 3901-3903.
50. Plow, E. F.; Haas, T. A.; Zhang, L.; Loftus, J.; Smith, J. W., Ligand binding to integrins. Journal of Biological Chemistry 2000, 275 (29), 21785-21788.
51. Ionov, L.; Minko, S., Mixed polymer brushes with locking switching. ACS applied materials & interfaces 2012, 4 (1), 483-489.
52. Sztilkovics, M.; Gerecsei, T.; Peter, B.; Saftics, A.; Kurunczi, S.; Szekacs, I.; Szabo, B.; Horvath, R., Single-cell adhesion force kinetics of cell populations from combined label-free optical biosensor and robotic fluidic force microscopy. Sci. Rep. 2020, 10 (1), 1-13.
53. Ladoux, B.; Nicolas, A., Physically based principles of cell adhesion mechanosensitivity in tissues. Rep. Prog. Phys. 2012, 75 (11), 1-25.
54. Sui, X. F.; Chen, Q.; Hempenius, M. A.; Vancso, G. J., Probing the Collapse Dynamics of Poly(N-isopropylacrylamide) Brushes by AFM: Effects of Co-nonsolvency and Grafting Densities. Small 2011, 7 (10), 1440-1447.
55. Sagvolden, G.; Giaever, I.; Pettersen, E. O.; Feder, J., Cell adhesion force microscopy. Proc. Natl. Acad. Sci. U.S.A. 1999, 96 (2), 471-476.
56. Patel, N. G.; Cavicchia, J. P.; Zhang, G.; Newby, B. M. Z., Rapid cell sheet detachment using spin-coated pNIPAAm films retained on surfaces by an aminopropyltriethoxysilane network. Acta Biomater. 2012, 8 (7), 2559-2567.
57. Yang, L.; Fan, X. G.; Zhang, J.; Ju, J., Preparation and Characterization of Thermoresponsive Poly(N-lsopropylacrylamide) for Cell Culture Applications. Polymers 2020, 12 (2), 1-24.
58. Nagase, K.; Yamato, M.; Kanazawa, H.; Okano, T., Poly(N-isopropylacrylamide)-based thermoresponsive surfaces provide new types of biomedical applications. Biomaterials 2018, 153, 27-48.
59. Doberenz, F.; Zeng, K.; Willems, C.; Zhang, K.; Groth, T., Thermoresponsive polymers and their biomedical application in tissue engineering—a review. J. Mater. Chem. B 2020, 8 (4), 607-628.
60. Ernst, O.; Lieske, A.; Jager, M.; Lankenau, A.; Duschl, C., Control of cell detachment in a microfluidic device using a thermo-responsive copolymer on a gold substrate. Lab Chip 2007, 7 (10), 1322-1329.
61. Tang, Z.; Akiyama, Y.; Itoga, K.; Kobayashi, J.; Yamato, M.; Okano, T., Shear stress-dependent cell detachment from temperature-responsive cell culture surfaces in a microfluidic device. Biomaterials 2012, 33 (30), 7405-7411.
62. Afif, S.; Ghaleh, H.; Nasiri, M.; Maher, B. M.; Abbasi, F., Adhesion, proliferation, and detachment of cells on poly (N-isopropyl acrylamide) brushes tethered on polystyrene using surface-initiated atom transfer radical polymerization. Mater. Today Commun. 2020, 25, 1-7.

1. A dynamic polymer surface comprising: a polymer layer having alternating micropatterns of adhesive domains and environmental stimuli-responsive repulsive domains,
the adhesive domains comprising one or more first polymer structures and having the characteristic of having an affinity for a target soft colloid particle, and
the repulsive domains comprising one or more second polymer structures that have the characteristic of being able to change form a retracted conformation to a swollen conformation in response to an environmental stimulus, such that application of the environmental stimulus activates the second polymer structures to the swollen conformation and does not activate the first polymer structures to a swollen conformation, such that the repulsive domains enlarge with respect to the adhesive domains.

2. The dynamic polymer surface of claim 1, wherein the polymer layer comprises polymer structures selected from the group consisting of: polymer brushes, grafted polymers, anchored polymers, polymer/polyelectrolyte multilayers, a polymer network, a polymer hydrogel thin film, and combinations thereof, wherein the first and second polymer structures are the same or different types of polymer structures.

3. The dynamic polymer surface of claim 1, wherein the affinity of the adhesive domains for the target soft colloidal particle selected from the group consisting of: binding affinity, size affinity, conformation affinity, and combinations thereof.

4. The dynamic polymer surface of claim 3, wherein the affinity comprises binding affinity and wherein first polymer structures comprise functional motifs having the characteristic of being complementary to and capable of reversibly binding complementary motifs on the target soft colloid particle.

5. The dynamic polymer surface of claim 4, wherein the functional motif comprises an RGD (Arg-Gly-Asp) motif.

6. The dynamic polymer surface of claim 1, wherein the target soft colloid particle is a biological soft colloid particle selected from the group consisting of lipid vesicles, cells, cellular organelles, protein clusters and complexes, polymer capsules, and microgel particles.

7. The dynamic polymer surface of claim 6, wherein the first and second polymer structures comprise biocompatible polymers.

8. The dynamic polymer surface of claim 1, wherein the environmental stimulus is selected from the group consisting of: temperature, pH, ionic strength, salinity, chemical concentration, light, magnetic field, electric field, ligand-protein interactions, mechanical forces or a combination thereof.

9. The dynamic polymer surface of claim 1, wherein the second polymer structures comprise a temperature sensitive polymer that changes conformation in response to a change in environmental temperature.

10. The dynamic polymer surface of claim 9, wherein the second polymer structures comprise poly(N-isopropylacrylamide) (PNIPAM) polymer brushes.

11. The dynamic polymer surface of claim 1, wherein a portion of the environmental stimuli-responsive repulsive domains are activatable super-repulsive domains comprising one or more third polymer structures that have the characteristic of being able to change conformation in response to a second environmental stimulus such that, upon application of the second environmental stimulus, the activatable super-repulsive domains swell and enlarge with respect to the adhesive domains and the environmental stimuli-responsive repulsive domains comprising the second polymer structures and have a greater surface height than the adhesive domains and the environmental stimuli-responsive repulsive domains comprising the second polymer structures, wherein the second environmental stimulus is different than the environmental stimulus that activates the second polymer structures.

12. A product comprising a substrate coated with the dynamic polymer surface of claim 1.

13. The product of claim 12, wherein the product is a cell culture system and further comprises a controlled environment in which the coated substrate is housed.

14. A method for non-enzymatically detaching particles from a polymer surface, the method comprising:
a) contacting the dynamic polymer surface of claim 1 with a liquid composition comprising target soft colloid particles with affinity for the adhesive domains in a controlled environment in for a first period of time during which the repulsive domains are in the retracted conformation; and
b) applying an activating environmental stimulus to the controlled environment for a second period of time to activate the second polymer structures to the swollen conformation such that the repulsive domains enlarge with respect to the adhesive domains, thereby physically contacting and exerting a mechanical force on particles adhered to the adhesive domains sufficient to detach a portion of particles from the adhesive domains.

15. The method of claim 14, wherein the liquid composition comprises target soft colloid particles and one or more non-target particles and the method comprises separating the target soft colloid particles from the non-target particles, wherein at least some of the non-target particles have a mild, non-specific affinity for the adhesive domains, and the target soft colloidal particles have a specific affinity for the adhesive domains that is stronger than the affinity of the non-target particles for the adhesive domain, such that applying the activating environmental stimulus to the controlled environment for the second period of time to activate the second polymer structures to the swollen conformation is effective to detach non-target particles from the adhesive domains while retaining target soft colloid particles adhered to the adhesive domains.

16. The method of claim 15, further comprising:
c) removing the activating environmental stimulus for a third period of time, such that the repulsive domains return to the retracted confirmation; and
d) repeating steps b and c for a number of cycles effective to increase the percent of target soft colloid particles bound to the adhesive domains.

17. The method of claim 16, further comprising:
e) removing the liquid composition from the controlled environment to retain bound target soft colloid particles on the dynamic polymer surface; and
f) releasing bound target soft colloid particles from the adhesive domains to obtain an enriched sample of target soft colloid particles.

18. The method of claim 14, wherein the activating environmental stimulus is a change in environmental temperature from a first temperature to a second temperature, wherein neither the first temperature nor the second temperature damages target particles.

19. The method of claim 14, wherein the liquid composition is a biological sample and the target soft colloid particles are selected from the group consisting of: lipid vesicles; cells, including rare cells; cellular organelles; protein clusters and complexes; polymer capsules; and
microgel particles.

20. The method of claim 14, wherein:
the method comprises growing and harvesting target cells;
the particles comprise a plurality of target cells with an affinity for the adhesive domains, and the liquid composition comprises a growth medium effective to grow and proliferate the plurality of target cells;
the first period of time is an amount of time effective for the target cells to proliferate to a desired amount of target cells;
applying the environmental stimulus to the controlled environment for a second period of time is effective to detach target cells from the adhesive domains; and
optionally harvesting the detached target cells from the controlled environment.
